(19) United States
(12) Patent Application Publication
(10) Pub. No.: US 2022/0031903 A1
(43) Pub. Date: Feb. 3, 2022

(54) ANTI-PATHOGENIC SOLUTION DISTRIBUTION SYSTEM

(71) Applicants: Arthur Victor Martin, Bluffton, SC (US); Kevin Michael Martin, Sunny Isles Beach, FL (US)

(72) Inventors: Arthur Victor Martin, Bluffton, SC (US); Kevin Michael Martin, Sunny Isles Beach, FL (US)

(21) Appl. No.: 17/304,404

(22) Filed: Jun. 21, 2021

Related U.S. Application Data
(60) Provisional application No. 62/706,137, filed on Aug. 3, 2020.

(51) Int. Cl. A61L 9/14

(52) U.S. Cl. CPC A61L 9/14; A61L 2209/16; A61L 2209/134; A61L 2209/11

(57) ABSTRACT

Solution distribution systems and methods of manufacturing and operating the same are provided. Solution distribution systems discussed herein are configured to dispense an anti-pathogenic solution into an air stream managed by an air handling system. Such solution distribution systems include a dispersion nozzle assembly structured to dispense the anti-pathogenic solution into the air stream in a dispersion pattern. The solution distribution systems also include a pump module configured to deliver the anti-pathogenic solution to the dispersion nozzle assembly at a dispersion pressure. The disclosed solution distribution systems further include a controller disposed in electrical communication with a control valve and the pump module. The controller is configured to engage the control valve to dispense the anti-pathogenic solution via the dispersion nozzle assembly into the air stream for a dispersion event period and at a dispersion interval.

### CROSS-REFERENCE TO RELATED APPLICATIONS

This application claims the benefit of U.S. Provisional Application No. 62/706,137 filed Aug. 3, 2020, the contents of which are incorporated by reference in its entirety for all purposes as if fully set forth herein.

### TECHNOLOGICAL FIELD

The present disclosure relates in general to solution distribution and more particularly to systems that are structured for dispensing an anti-pathogenic solution into an air stream.

### BACKGROUND

The average adult breathes in excess of 50 cubic meters of air per 24-hour period. Air can often, unbeknownst to humans, contain variable amounts of viruses, fungi, bacteria, yeasts, plant pollen, and other harmful disease carrying particulates. Indoor settings, such as offices, especially present problems as the same potentially harmful air is often recirculated by air rotation units. Through applied effort, ingenuity, and innovation, many of these identified problems have been solved by developing solutions that are included in embodiments of the present invention, many examples of which are described in detail herein.

### BRIEF SUMMARY

The following presents a simplified summary in order to provide a basic understanding of some aspects of the present disclosure. This summary is not an extensive overview and is intended to neither identify key or critical elements nor delineate the scope of such elements. Its purpose is to present some concepts of the described features in a simplified form as a prelude to the more detailed description that is presented later.

In various embodiments, solution distribution systems and methods of manufacturing and operating the same are provided. In an example embodiment, a solution distribution system configured to dispense an anti-pathogenic solution into an air stream managed by an air handling system is provided. The solution distribution system includes a dispersion nozzle assembly structured to dispense the anti-pathogenic solution into the air stream in a dispersion pattern. The solution distribution system also includes a pump module configured to deliver the anti-pathogenic solution to the dispersion nozzle assembly at a dispersion pressure. The solution distribution system further includes a controller disposed in electrical communication with a control valve and the pump module. The controller is configured to engage the control valve to dispense the anti-pathogenic solution via the dispersion nozzle assembly into the air stream for a dispersion event period and at a dispersion interval.

In some embodiments, the solution distribution system also includes a pick-up valve and defines a solution flow path between the pick-up valve and the dispersion nozzle assembly. In some embodiments, the control valve is a three-way valve connected to a suction hose, a delivery hose, and a bleed hose, and wherein the solution flow path is defined to proceed from the pick-up valve, through the suction hose and delivery hose, and to the dispersion nozzle assembly. In some embodiments, the control valve is the pick-up valve. In some embodiments, the solution distribution system also includes a backflow pressure regulator disposed along the solution flow path between the control valve and the dispersion nozzle assembly.

In some embodiments, the backflow pressure regulator is structured to limit air introduction into the solution flow path. In some embodiments, the solution distribution system includes a fluid pickup assembly configured to draw the anti-pathogenic solution from a solution container and defines a solution flow path between a pick-up valve and the dispersion nozzle assembly.

In some embodiments, the fluid pickup assembly includes a strainer module and a pick-up valve. In some embodiments, the strainer module of the fluid pickup assembly defines a weighted, cylindrical filter body structured to limit sediment within the solution container from entering the solution flow path. In some embodiments, the solution distribution system includes an enclosure body structured to enclose the pump module and the controller. In such an embodiment, the enclosure body, the pump module, and the controller combine to define a main enclosure weight of less than 5 kilograms.

In some embodiments, the dispersion event period and dispersion pressure are selected to deliver a dispersion event dosage of anti-pathogenic solution between approximately 1.2 fluid ounces and approximately 1.5 fluid ounces into the air stream. In some embodiments, the dispersion event period is from approximately 6 seconds to approximately 8 seconds.

In some embodiments, the dispersion pressure is between approximately 50 PSI and approximately 55 PSI. In some embodiments, the dispersion interval is approximately one hour. In some embodiments, the dispersion event period and the dispersion event interval are selected to distribute over ten million anti-pathogenic solution molecules into the air stream over a treatment period of between approximately 6 and approximately 8 hours. In some embodiments, the dispersion interval is between approximately 30 minutes and approximately 90 minutes.

In some embodiments, the dispersion nozzle assembly is structured to produce an angular fan dispersion pattern of anti-pathogenic solution. In some embodiments, the dispersion event period, the angular fan dispersion pattern, and the dispersion event interval are selected to distribute over ten million anti-pathogenic solution molecules into the air stream over a treatment period of between approximately 6 and approximately 8 hours.

In some embodiments, the dispersion nozzle assembly is structured to distribute the anti-pathogenic solution in the angular fan dispersion pattern at a molecule size of between approximately 8 and approximately 15 microns. In some embodiments, the anti-pathogenic solution is non-metallic, has a pH value of between approximately 1.5 and approximately 1.6, and a specific gravity of between approximately 1.10 and approximately 1.30.

In some embodiments, the air handling system is an air rotation unit. In some embodiments, the dispersion nozzle assembly is structured for mounting upstream of a heating or cooling coil of the air rotation unit. In some embodiments, the solution distribution system also includes an enclosure body structured to enclose the pump module and the controller, and the enclosure body is configured for mounting to an exterior wall of the air rotation unit. In some embodiments, the dispersion nozzle assembly is structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

In another example embodiment, a method of assembling a solution distribution system configured to dispense an anti-pathogenic solution into an air stream managed by an air handling system is provided. The method includes providing a dispersion nozzle assembly structured to dispense the anti-pathogenic solution into the air stream in a dispersion pattern. The method also includes coupling a pump module to the dispersion nozzle assembly. The pump module is configured to deliver the anti-pathogenic solution to the dispersion nozzle assembly at a dispersion pressure. The method also includes providing a controller disposed in electrical communication with a control valve and the pump module. The controller is configured to engage the control valve to dispense the anti-pathogenic solution via the dispersion nozzle assembly into the air stream for a dispersion event period and at a dispersion interval.

In some embodiments, the method also includes providing a pick-up valve coupled to the pump module and further defining a solution flow path between the pick-up valve and the dispersion nozzle assembly. In some embodiments, the control valve is a three-way valve connected to a suction hose, a delivery hose, and a bleed hose, and wherein the solution flow path is defined to proceed from the pick-up valve, through the suction hose and delivery hose, and to the dispersion nozzle assembly. In some embodiments, the control valve is the pick-up valve.

In some embodiments, the method also includes providing a backflow pressure regulator disposed along the solution flow path between the control valve and the dispersion nozzle assembly. In some embodiments, the backflow pressure regulator is structured to limit air introduction into the solution flow path.

In some embodiments, the method also includes fluidly coupling a fluid pickup assembly to a solution flow path. In such an embodiment, the fluid pickup assembly is configured to draw the anti-pathogenic solution from a solution container and the solution flow path is defined between a pick-up valve and the dispersion nozzle assembly.

In some embodiments, the fluid pickup assembly includes a strainer module and a pick-up valve. In some embodiments, the strainer module of the fluid pickup assembly defines a weighted, cylindrical filter body structured to limit sediment within the solution container from entering the solution flow path.

In some embodiments, the method also includes enclosing the pump module and the controller within an enclosure body. In such an embodiment, the enclosure body, the pump module, and the controller combine to define a main enclosure weight of less than 5 kilograms.

In some embodiments, the dispersion event period and the dispersion pressure are selected to deliver a dispersion event dosage of anti-pathogenic solution between approximately 1.2 fluid ounces and approximately 1.5 fluid ounces into the air stream. In some embodiments, the dispersion event period is from approximately 6 seconds to approximately 8 seconds. In some embodiments, the dispersion pressure is between approximately 50 PSI and approximately 55 PSI.

In some embodiments, the dispersion interval is approximately one hour. In some embodiments, the dispersion event period and dispersion event interval are selected to distribute over ten million anti-pathogenic solution molecules into the air stream over a treatment period of between approximately 6 and approximately 8 hours. In some embodiments, the dispersion interval is between approximately 30 minutes and approximately 90 minutes. In some embodiments, the method includes structuring the dispersion nozzle assembly to produce an angular fan dispersion pattern of anti-pathogenic solution.

In some embodiments, the dispersion event period, the angular fan dispersion pattern, and the dispersion event interval are selected to distribute over ten million anti-pathogenic solution molecules into the air stream over a treatment period of between approximately 6 and approximately 8 hours. In some embodiments, the dispersion nozzle assembly is structured to distribute the anti-pathogenic solution in the angular fan dispersion pattern at a molecule size of between approximately 8 and approximately 15 microns.

In some embodiments, the anti-pathogenic solution is non-metallic, has a pH value of between approximately 1.5 and approximately 1.6, and a specific gravity of between approximately 1.10 and approximately 1.30. In some embodiments, the air handling system is an air rotation unit. In some embodiments, the dispersion nozzle assembly is structured for mounting upstream of a heating or cooling coil of the air rotation unit. In some embodiments, the method also includes enclosing the pump module and the controller within an enclosure body. In such an embodiment, the enclosure body is configured for mounting to an exterior wall of the air rotation unit. In some embodiments, the dispersion nozzle assembly is structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

In another example embodiment, a method of dispensing an anti-pathogenic solution into an air stream managed by an air handling system is provided. The method includes causing a first dispersion event via a dispersion nozzle assembly. The first dispersion event provides a dispersion event dosage of anti-pathogenic solution for a dispersion event period. The method also includes causing a second dispersion event via the dispersion nozzle assembly at a dispersion interval. The second dispersion event provides the dispersion event dosage of anti-pathogenic solution for the dispersion event period.

In some embodiments, the method also includes engaging a control valve to dispense the anti-pathogenic solution via the dispersion nozzle assembly into the air stream for the dispersion event period.

In some embodiments, the method also includes causing one or more additional dispersion events at the dispersion interval after the second dispersion event. In some embodiments, the dispersion event dosage is provided at a dispersion pressure. In some embodiments, the dispersion event period and the dispersion pressure are selected to deliver the dispersion event dosage of the anti-pathogenic solution between approximately 1.2 fluid ounces to approximately 1.5 fluid ounces.

In some embodiments, the dispersion event period is from approximately 6 seconds to approximately 8 seconds. In some embodiments, the dispersion pressure is between approximately 50 PSI and approximately 55 PSI. In some embodiments, the dispersion interval is approximately one hour. In some embodiments, the dispersion event period and the dispersion event interval are selected to distribute over ten million anti-pathogenic solution molecules into the air stream over a treatment period of between approximately 6 and approximately 8 hours.

In some embodiments, the dispersion interval is between approximately 30 minutes and approximately 90 minutes. In some embodiments, the dispersion nozzle assembly is structured to produce the dispersion event dosage at an angular fan dispersion pattern of anti-pathogenic solution. In some embodiments, the dispersion event period, the angular fan dispersion pattern, and the dispersion event interval are selected to distribute over ten million anti-pathogenic solution molecules into the air stream over a treatment period of between approximately 6 and approximately 8 hours.

In some embodiments, the dispersion nozzle assembly is structured to distribute the anti-pathogenic solution in the angular fan dispersion pattern at a molecule size of between approximately 8 and approximately 15 microns. In some embodiments, the anti-pathogenic solution is non-metallic, has a pH value of between approximately 1.5 and approximately 1.6, and a specific gravity of between approximately 1.10 and approximately 1.30.

In some embodiments, the air handling system is an air rotation unit. In some embodiments, the dispersion nozzle assembly is structured for mounting upstream of a heating or cooling coil of the air rotation unit. In some embodiments, the dispersion nozzle assembly is structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

In still another example embodiment, a computer program product is provided. The computer program product includes at least one non-transitory computer-readable storage medium having computer-executable program code portions stored therein. The computer-executable program code portions include program code instructions configured to dispense an anti-pathogenic solution into an air stream managed by an air handling system. The computer-executable program code portions including program code instructions are configured to cause a first dispersion event via a dispersion nozzle assembly. The first dispersion event provides a dispersion event dosage of anti-pathogenic solution for a dispersion event period. The computer-executable program code portions including program code instructions are also configured to cause a second dispersion event via the dispersion nozzle assembly at a dispersion interval. The second dispersion event provides the dispersion event dosage of anti-pathogenic solution for the dispersion event period.

In some embodiments, the computer program product also includes program code instructions configured to engage a control valve to dispense the anti-pathogenic solution via the dispersion nozzle assembly into the air stream for the dispersion event period. In some embodiments, the computer program product also includes program code instructions to cause one or more additional dispersion events at the dispersion interval after the second dispersion event.

In some embodiments, the dispersion event dosage is provided at a dispersion pressure. In some embodiments, the dispersion event period and dispersion pressure are selected to deliver the dispersion event dosage of the anti-pathogenic solution between approximately 1.2 fluid ounces to approximately 1.5 fluid ounces. In some embodiments, the dispersion event period is from approximately 6 seconds to approximately 8 seconds.

In some embodiments, the dispersion pressure is between approximately 50 PSI and approximately 55 PSI. In some embodiments, the dispersion interval is approximately one hour. In some embodiments, the dispersion event period and dispersion event interval are selected to distribute over ten million anti-pathogenic solution molecules into the air stream over a treatment period of between approximately 6 and approximately 8 hours.

In some embodiments, the dispersion interval is between approximately 30 minutes and approximately 90 minutes. In some embodiments, the dispersion nozzle assembly is structured to produce the dispersion event dosage at an angular fan dispersion pattern of anti-pathogenic solution. In some embodiments, the dispersion event period, the angular fan dispersion pattern, and the dispersion event interval are selected to distribute over ten million anti-pathogenic solution molecules into the air stream over a treatment period of between approximately 6 and approximately 8 hours.

In some embodiments, the dispersion nozzle assembly is structured to distribute the anti-pathogenic solution in the angular fan dispersion pattern at a molecule size of between approximately 8 and approximately 15 microns. In some embodiments, the anti-pathogenic solution is non-metallic, has a pH value of between approximately 1.5 and approximately 1.6, and a specific gravity of between approximately 1.10 and approximately 1.30.

In some embodiments, the air handling system is an air rotation unit. In some embodiments, the dispersion nozzle assembly is structured for mounting upstream of a heating or cooling coil of the air rotation unit. In some embodiments, the dispersion nozzle assembly is structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

In still another example embodiment, a dispersion nozzle assembly configured to dispense an anti-pathogenic solution into an air stream managed by an air handling system is provided. The nozzle assembly includes a dispersion nozzle defining a nozzle outlet structured to dispense the anti-pathogenic solution into the air stream in a dispersion pattern. The dispersion nozzle assembly also includes a backflow pressure regulator disposed along a solution flow path to the dispersion nozzle. The backflow pressure regulator is structured to limit air introduction into the solution flow path.

In some embodiments, the solution flow path is defined between the dispersion nozzle and a pump module. In such an embodiment, the pump module is configured to provide to the dispersion nozzle at a dispersion pressure. In some embodiments, the dispersion nozzle is from approximately 12 inches to approximately 18 inches wide. In some embodiments, the dispersion pressure at the nozzle outlet is between approximately 50 PSI and approximately 55 PSI. In some embodiments, the dispersion pattern is a pulsating spray.

In some embodiments, the dispersion nozzle is made of stainless steel. In some embodiments, the dispersion nozzle is fluidly coupled to the pump module via a delivery hose. In some embodiments, the dispersion nozzle assembly is in communication with a control valve engaged by a controller configured to engage a control valve to dispense the anti-pathogenic solution via the dispersion nozzle assembly into the air stream for a dispersion event period and at a dispersion interval.

In some embodiments, the dispersion interval is approximately 60 minutes. In some embodiments, the dispersion interval is between approximately 30 minutes and approximately 90 minutes. In some embodiments, the dispersion event period is from approximately 6 seconds to approximately 8 seconds. In some embodiments, the dispersion event period and dispersion event interval are selected to distribute over ten million anti-pathogenic solution molecules into the air stream over a treatment period of between approximately 6 and approximately 8 hours.

In some embodiments, the air handling system is an air rotation unit. In some embodiments, the dispersion nozzle is structured for mounting upstream of a heating or cooling coil of the air rotation unit. In some embodiments, based on the size of the dispersion nozzle, the dispersion event period and dispersion pressure are selected to deliver a dispersion event dosage of anti-pathogenic solution between approximately 1.2 fluid ounces and approximately 1.5 fluid ounces into the air stream.

In some embodiments, the dispersion pattern is an angular fan dispersion pattern of the anti-pathogenic solution. In some embodiments, the dispersion event period, the angular fan dispersion pattern, and the dispersion event interval are selected to distribute over ten million anti-pathogenic solution molecules into the air stream over a treatment period of between approximately 6 and approximately 8 hours. In some embodiments, the dispersion nozzle assembly is structured to distribute the anti-pathogenic solution in the angular fan dispersion pattern at a molecule size of between approximately 8 and approximately 15 microns.

In some embodiments, the anti-pathogenic solution is non-metallic, has a pH value of between approximately 1.5 and approximately 1.6, and a specific gravity of between approximately 1.10 and approximately 1.30. In some embodiments, the dispersion nozzle assembly is structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

The above summary is provided merely for purposes of summarizing some example embodiments to provide a basic understanding of some aspects of the invention. Accordingly, it will be appreciated that the above-described embodiments are merely examples and should not be construed to narrow the scope or spirit of the invention in any way. It will be appreciated that the scope of the invention encompasses many potential embodiments in addition to those here summarized, some of which will be further described below.

### BRIEF DESCRIPTION OF THE DRAWINGS

Having thus described the disclosure in general terms, reference will now be made to the accompanying drawings, which are not necessarily drawn to scale, and wherein:

FIG. 1A illustrates a solution distribution system structured in accordance with various embodiments of the present disclosure;

FIG. 1B illustrates a detail view of a pump enclosure assembly for a solution distribution system structured in accordance with various embodiments of the present disclosure;

FIG. 2A illustrates a schematic diagram of a solution distribution system structured in accordance with various embodiments of the present disclosure;

FIG. 2B is an exploded view of an example solution distribution system structured in accordance with various embodiments of the present disclosure;

FIG. 3A illustrates an example enclosure base of an example solution distribution system structured in accordance with various embodiments of the present disclosure;

FIG. 3B illustrates a perspective view of an example enclosure top plate of an example solution distribution system structured in accordance with various embodiments of the present disclosure;

FIG. 3C illustrates a perspective view of an example enclosure cover plate of an example solution distribution system structured in accordance with various embodiments of the present disclosure;

FIG. 4 illustrates an example enclosure base of an example solution distribution system structured in accordance with various embodiments of the present disclosure;

FIG. 5 illustrates an example dispersion valve assembly used in an example solution distribution system structured in accordance with various embodiments of the present disclosure;

FIG. 6A illustrates a top view of an example dispersion valve assembly, such as a three-way valve, used in an example solution distribution system structured in accordance with various embodiments of the present disclosure;

FIG. 6B illustrates a cross-sectional view of an example dispersion valve assembly, such as a three-way valve, used in an example solution distribution system structured in accordance with various embodiments of the present disclosure;

FIG. 7A illustrates an example fluid pickup assembly used in an example solution distribution system structured in accordance with various embodiments of the present disclosure;

FIG. 7B illustrates various orthogonal views of an example pick-up valve used in a fluid pickup assembly structured in accordance with various embodiments of the present disclosure;

FIG. 7C illustrates a cross-sectional view of an example pick-up valve used in a fluid pickup assembly structured in accordance with various embodiments of the present disclosure;

FIG. 8A illustrates a regulator base used in a backflow pressure regulator structured in accordance with various embodiments of the present disclosure;

FIG. 8B illustrates a cross-sectional view of the regulator base shown in FIG. 8A structured in accordance with various embodiments of the present disclosure;

FIG. 8C illustrates various orthogonal views of an example regulator cover used in a backflow pressure regulator structured in accordance with various embodiments of the present disclosure;

FIG. 8D illustrates a cross-sectional view of the regulator cover shown in FIG. 8C structured in accordance with various embodiments of the present disclosure;

FIG. 8E illustrates an example dispersion nozzle assembly in accordance with various embodiments of the present disclosure;

FIGS. 9A and 9B illustrate a dispersion nozzle head used in a dispersion nozzle assembly structured in accordance with various embodiments of the present disclosure;

FIG. 10 illustrates a circuit board box in structured in accordance with various embodiments of the present disclosure;

FIG. 11 illustrates an example schematic diagram of a controller structured in accordance with various embodiments of the present disclosure;

FIG. 12A illustrates an example suction hose assembly structured in accordance with various embodiments of the present disclosure;

FIG. 12B illustrates a delivery hose assembly structured in accordance with various embodiments of the present disclosure;

FIG. 13A illustrates an example air handling system that may be configured to receive an example solution distribution system installed in accordance with various embodiments of the present disclosure;

FIG. 13B illustrates an example enclosure body of an example solution distribution system as installed on the exterior of the air handling system shown in FIG. 13 A;

FIG. 14A illustrates a dispersion nozzle assembly of an example solution distribution system mounted on the interior of an air handling system in accordance with the present disclosure;

FIG. 14B illustrates a dispersion nozzle assembly of an example embodiment mounted at another location on the interior of an air handling system in accordance with the present disclosure;

FIG. 15 is a flow chart illustrating a method of assembling a solution dispersion system of various embodiments in accordance with the present disclosure; and

FIG. 16 is a flow chart illustrating a method of dispensing an anti-pathogenic solution into an air stream in accordance with various embodiments of the present disclosure.

### DETAILED DESCRIPTION

The present invention now will be described more fully hereinafter with reference to the accompanying drawings in which some but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout. As used herein, terms such as “front,” “rear,” “top,” etc. are used for explanatory purposes in the examples provided below to describe the relative position of certain components or portions of components. For example, the term “top edge” may be used to describe an edge of a component; however, the edge may be on the top, bottom, or side, depending on the orientation of the particular component being described. As used herein, the terms ‘substantially’ and ‘approximately’ refer to tolerances within manufacturing and/or engineering standards.” As used herein, the terms “solution” and “anti-pathogenic solution” both refer to the anti-pathogenic solution used with the solution distribution system discussed herein.

Maintaining adequate air quality, especially indoors is important to sustaining wellness and health for humans. Viruses that spread easily, such as COVID-19, are difficult to contain especially indoors, such as an office or industrial setting, when carried about by air conditioners and other air handling systems. Various embodiments discussed herein detail solution distribution systems that are configured to address the threat posed by viruses and other airborne threats to human health by dispensing an anti-pathogenic solution into air streams managed by indoor air handling systems.

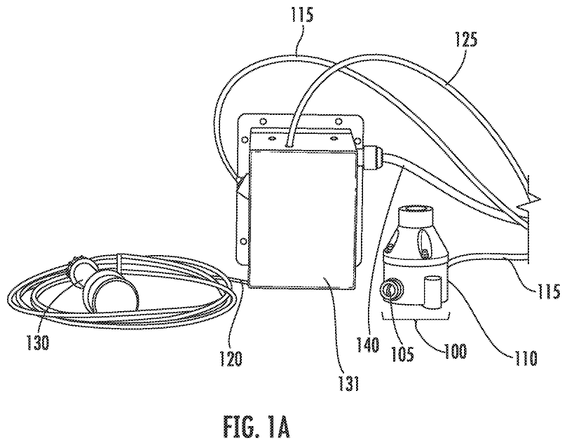

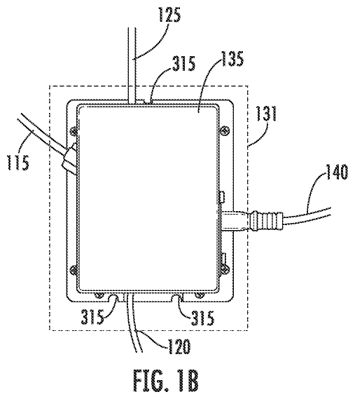

FIG. 1A depicts a solution distribution system 10 configured to dispense an anti-pathogenic solution into an air stream managed by an air handling system in accordance with various embodiments. The depicted solution distribution system 10 includes a dispersion nozzle assembly 100, a pump enclosure assembly 131, and a pick-up valve 130. The pump enclosure assembly 131 is connected to the pick-up valve 130 by a suction hose 120 and to the dispersion nozzle assembly 100 by a delivery hose 115. A bleed hose 125 also extends from the pump enclosure assembly 131 as shown.

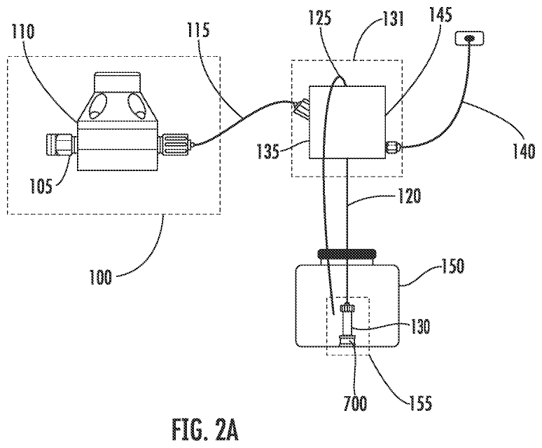

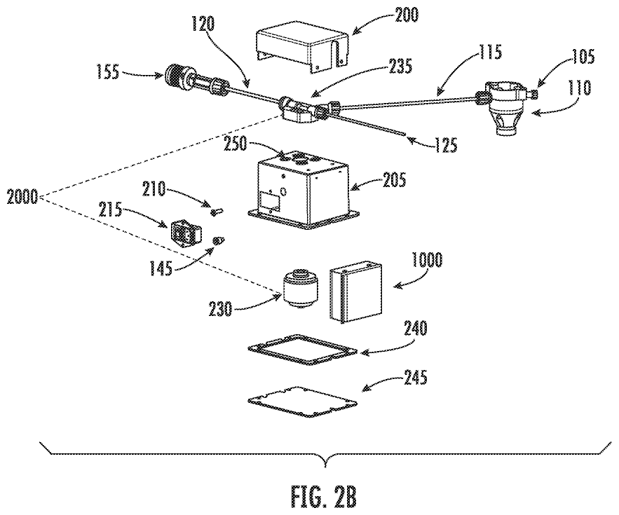

FIG. 1B provides a detail view of the pump enclosure assembly 131, which includes an enclosure body 135 that is structured to enclose and protect a pump module 2000 and a controller 220 as discussed in reference to FIG. 2B below. The depicted pump enclosure assembly 131 and its internal components (e.g., the dispersion valve assembly 235, the pump 230, and the controller 220) define a main enclosure weight of less than five kilograms.

The pump module 2000 includes a dispersion valve assembly 235 and a pump 230 as discussed below in reference to FIG. 2B. In some embodiments, the dispersion valve assembly 235 is a three-way valve and operates as a control valve for the solution distribution system 10. Each of the delivery hose 115, suction hose 120, and the bleed hose 125 discussed herein is connected to the dispersion valve assembly 235 (e.g., the three-way valve). In various embodiments, during operation, the pump 230 draws the solution to move along a solution flow path from the pick-up valve 130 through the suction hose 120, continuing through the pump module 2000 (e.g., the dispersion valve assembly 235) into the delivery hose 115 and into the dispersion nozzle assembly 100, ultimately being dispensed via the dispersion nozzle 105.

The depicted enclosure body 135 includes a power switch (not shown) and is connected to a power cord 140 that connects the solution distribution system 10 to a power source. Alternatively or additionally, the solution distribution system 10 may include a dedicated power supply (e.g., a power supply, such as a battery, may be disposed either within or adjacent to an enclosure body 135).

The depicted enclosure body 135 defines enclosure mountings 315 that are configured to facilitate installation of the enclosure body 135 within or proximate to an air handling system. For example, the depicted enclosure body 135 includes three enclosure mountings 315 that define apertures configured to receive a screw or other fastener to fix the enclosure body 135 in place.

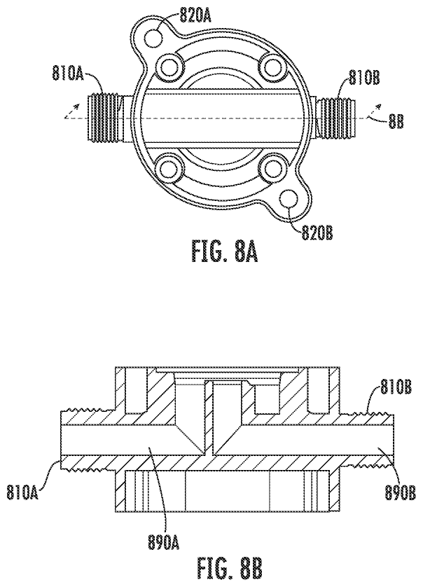

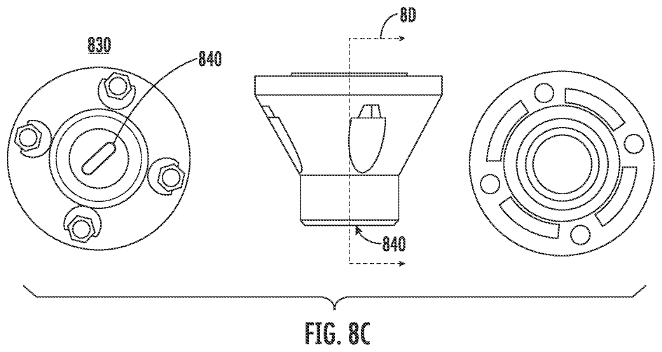

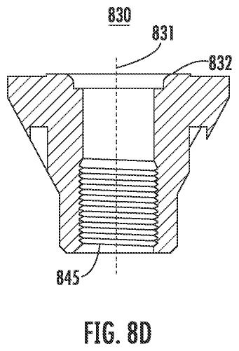

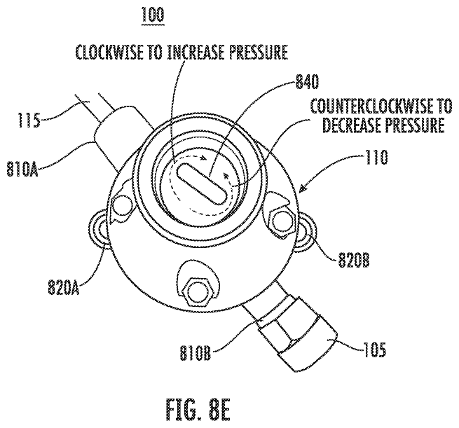

The dispersion nozzle assembly 100 shown in FIG. 1A includes a dispersion nozzle 105 and a backflow pressure regulator 110. In various embodiments, the dispersion nozzle assembly 100 is fluidly coupled to a pump module 2000 (housed within the enclosure body 135 in various embodiments, such as shown in FIGS. 1A and 1B) via a delivery hose 115. The backflow pressure regulator 110 is configured to maintain a dispersion pressure upstream of itself within the delivery hose 115. As discussed in reference to FIGS. 8A-8E, the depicted backflow pressure regulator 110 is structured for adjustment by a user or operator to change the pressure of the solution being provided to the dispersion nozzle 105. For example, the pump module 2000 may cause the pressure of the solution therein to rise to or over a given dispersion pressure and the backflow pressure regulator 110 may be adjusted to maintain the desired dispersion pressure. In some embodiments, the backflow pressure regulator 110 may be adjusted to account for changes to the system that might otherwise undesirably alter the dispersion pressure, such as elevated positioning of the dispersion nozzle assembly 100 relative to the pump enclosure assembly 131.

In some embodiments, the controller is configured to engage the pump module 2000 to cause pressure within the delivery hose 115 to exceed a setpoint (e.g., a dispersion pressure) selected for the backflow pressure regulator 110 thereby causing a valve (not shown) within the backflow pressure regulator 110 to open and release the excess pressure in a dispersion event.

The solution distribution system 10 is configured such that anti-pathogenic solution travels along the delivery hose 115 (from the pump module 2000) through the backflow pressure regulator 110 and into the dispersion nozzle 105. The depicted delivery hose 115 is made out of a high-density polyethylene (HDPE) tubing material. In an example embodiment, the delivery hose 115 may be approximately 7620 millimeters (approximately 25 feet) long. In some embodiments, the delivery hose 115 may define an inner diameter of 4 millimeters and an outer diameter of 6 millimeters.

The pick-up valve 130 depicted in FIG. 1A is fluidly coupled to the pump module 2000 (housed within the enclosure body 135 in various embodiments, such as those shown in FIGS. 1A and 1B) via a suction hose 120. The suction hose 120 may be a polyurethane tubing material. In an example embodiment, the suction hose 120 may be approximately 2438.4 millimeters (approximately 8 feet) long. In some embodiments, the suction hose 120 defines an inner diameter of 4 millimeters and an outer diameter of 6 millimeters. The suction hose 120 may be clear or translucent in some embodiments.

The depicted solution distribution system 10 further includes a bleed hose 125 that is configured with a purge feature that purges the system of excess air or to drain the system of antipathogenic solution for system cleaning. The purge feature activates a purge cycle that removes air and/or solution from the solution flow path. The bleed hose 125 may return any solution removed from the solution flow path to the solution container 150. In various embodiments, the purge feature may be activated automatically (e.g., periodically or in an instance the system monitors one or more sensors that are configured to determine excess amounts of air in the system). Alternatively or additionally, the purge feature may be activated manually (e.g., such as via a purge button 145 shown in the example embodiments shown in FIGS. 2A and 2B). While a bleed hose 125 is used, various other types of bleeding methods may be used in various embodiments (e.g., the solution distribution system may have a bleed valve in place of the bleed hose). The bleed hose 125 may be made out of a high density polyethylene (HDPE) tubing material. In an example embodiment, the bleed hose 125 may be approximately 3048 millimeters (Approximately 10 feet) long. The bleed hose 125 may define an inner diameter of 4 millimeters and an outer diameter of 6 millimeters.

Referring now to FIG. 2A, the depicted dispersion nozzle assembly 100 is in communication with the fluid pickup assembly 155 via a solution flow path. Said differently, antipathogenic solution is drawn through the fluid pickup assembly 155 and moves through the depicted solution distribution system 10 along a solution flow path until it is dispensed into an air stream by the dispersion nozzle assembly 100. In various embodiments, the solution flow path is defined by various fluid handling components of the solution distribution system 10 including the pick-up valve 130, the suction hose 120, the pump module 2000 (housed within the enclosure body 135), the delivery hose 115, and the dispersion nozzle assembly 100, including at least the backflow pressure regulator 110, and the dispersion nozzle 105.

The depicted fluid pickup assembly 155 is positioned within solution container 150 and includes the pick-up valve 130 and a strainer module 700. In various embodiments, the pick-up valve 130 is structured to be disposed within the solution container 150, such that upon activation of the pick-up valve 130, the solution is transported along the solution flow path. As discussed below in more detail, the fluid pickup assembly 155 may also include a strainer configured to limit sediment that might be present within the solution container from entering the solution flow path.

The depicted solution container 150 is a five-gallon container. The size of the solution container 150 may determine the amount new solution that is required to be added to the solution container 150 (e.g., a five-gallon container may last approximately 3.5 weeks). A solution level sensor (not shown) may be provided to monitor the amount of solution in the solution container 150. The solution level sensor may be internal to the fluid pickup assembly 155 (e.g., the solution level sensor may be disposed within the fluid pickup assembly 155). Alternatively, the solution level sensor may be external to the fluid pickup assembly 155 (e.g., disposed within the solution container 150). The solution level sensor may monitor the amount of solution remaining in the solution container (e.g., the solution level sensor may notify a user when the solution is below a threshold amount of solution).

The bleed hose 125 may be structured to remove air and/or solution from the solution flow path. The bleed hose 125 may deposit the removed air and/or solution into the solution container 150. For example, the bleed hose 125 may be attached to the dispersion valve assembly 235 at a first end and the second end may be located in the solution container 150. The bleed hose being routed to the solution container 150 allows any solution taken from the solution flow path by the bleed hose 125 may be returned to the system (e.g., allows for zero loss while also purging the system). The solution distribution system 10 may be configured to purge the solution for an extended period of in certain instances (e.g., a power outage). In such an instance, more than air (e.g., solution) may be removed by the solution flow path via the bleed hose 125. Alternatively, the bleed hose 125 may be routed outside of the solution container 150 (e.g., either to another container to preserve solution or to elsewhere).

In various embodiments, the solution remaining in the solution flow path between the pump module 2000 and dispersion nozzle 105 and not dispersed during the activation cycle remains in the solution flow path. As such, air is not drawn into the fluid supply line allowing for full solution dispersion during each subsequent limited activation cycle thereby providing full efficacy to the indoor air. However, in some instances, air may enter the solution flow path. Such air can be removed via the purge feature discussed herein.

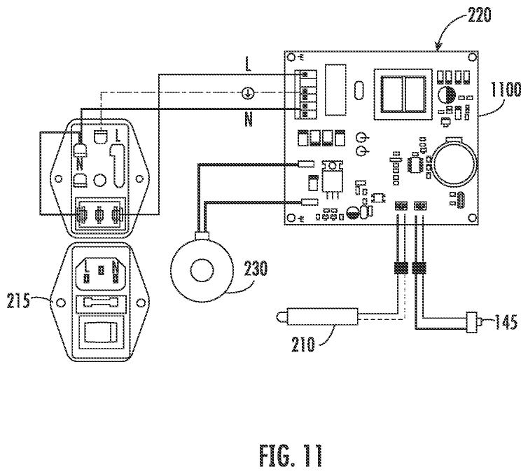

As discussed above, a purge button 145 may be provided to allow for the activation of a system purge (e.g., remove the air from the solution fluid path via the bleed hose 125). In some embodiments, the purge button 145 may in electrical communication with the controller 220 (shown in FIG. 11) in order to activate the dispersion valve assembly 235 of the pump module 2000 to remove any air in the solution flow path.

The purge cycle, whether activated by engaging the purge button 145 or otherwise, may remove air and/or solution from the system for a predetermined amount of time via the bleed hose 125. The purge cycle may be activated to remove any air in the solution distribution system 10, though the purge cycle may be sufficiently long to also remove some amount of solution (e.g., the bleed hose 125 may remove solution from the system once all of the air has been removed). As discussed above, the bleed hose 125 may be positioned to return any solution removed to the solution container 150. The purge cycle may be activated for approximately 20 seconds in an example embodiment.

Referring now to FIG. 2B, an exploded view of a solution distribution system 10 of various embodiments is shown. Various embodiments of the solution distribution system 10 may include different components and/or structure of components.

The depicted enclosure body 135 includes an enclosure top plate 200, an enclosure base 205, and an enclosure cover plate 245. In various embodiments, a sealing gasket 240 may be provided between the enclosure base 205 and the enclosure cover plate 245. While the enclosure body 135 shown in FIG. 2B includes the enclosure top plate 200, the enclosure base 205, and the enclosure cover plate 245, various example enclosure bodies may include more or less individual components. For example, a single enclosure base 205 may be provided that is structured to enclose all of the internal pump and control valve components but without the enclosure top plate 200 and the enclosure cover plate 245.

The depicted enclosure body 135 is comprised of multiple pieces (e.g., the enclosure top plate 200, enclosure base 205, and the enclosure base 205) to allow for service access to certain components disposed within the enclosure body 135 (e.g., the enclosure top plate 200 may be removed to access the dispersion valve assembly 235 without having to access the enclosure base 205).

The pump module 2000 includes a dispersion valve assembly 235. In some embodiments, the dispersion valve assembly 235 is a three-way valve. Each of the delivery hose 115, suction hose 120, and the bleed hose 125 discussed herein is connected to the dispersion valve assembly 235 (e.g., the three-way valve). In various embodiments, during operation, the solution moves along a solution flow path from the pick-up valve 130 through the suction hose 120, continuing through the pump module 2000 into the delivery hose 115 and into the dispersion nozzle assembly 100, ultimately being dispensed via the dispersion nozzle 105.

The pump module 2000 also includes the pump 230. The depicted pump 230 of the pump module 2000 and the controller 220 (not shown) is enclosed between the enclosure base 205 and the enclosure cover plate 245. The controller 220 may be housed within an additional casing as shown (e.g., the controller 220 is enclosed within the circuit board enclosure 1000 in FIG. 2B). The dispersion valve assembly 235 (e.g., three-way valve) of the pump module 2000 may be disposed between the enclosure base 205 and the enclosure top plate 200. The depicted enclosure base 205 includes one or more pump apertures 250 configured to allow communication between the pump 230 of the pump module 2000 and the dispersion valve assembly 235 (e.g., three-way valve) of the pump module 2000. In some embodiments, the pump 230 is configured to manipulate one or more valves of the dispersion valve assembly 235 to cause fluid to be drawn into the delivery hose 115 or the bleed hose 125. In other embodiments, the controller 220 is configured to control one or more valves of the dispersion valve assembly 235 directly without using the pump 230 as an intermediary.

In the depicted embodiment, the controller 220 (shown in FIG. 11) is configured to operate the pump module 2000 (e.g., the pump 230 and the dispersion valve assembly 235). Additionally, the controller 220 is configured to activate various other operations of the solution distribution system 10. The controller 220 may constitute all or substantially all of the power draw (e.g., the controller 220 determines the amount of amp draw at a given time) for the system. The amp draw may be approximately zero (or extremely low) during dispersion intervals (e.g., time windows defined between dispersion event periods). In some embodiments, the amp draw during the activation of the solution distribution system 10 for dispersion event periods is approximately 0.8 amps.

The depicted pump 230 of the pump module 2000 is in communication with the solution flow path, such that the pump 230 draws the solution from the pick-up valve 130 through the dispersion valve assembly 235 (e.g., the three-way valve), and into the dispersion nozzle assembly 100. The pump 230 may be configured to enable the solution distribution system 10 to have a minimum flow rate of 0.00113 gallons per hour and a maximum flow rate of 0.001239 gallons per hour.

In various embodiments, an indicator light 210, a purge button 145, and/or a power switch 215 are provided within the enclosure body (e.g., within the enclosure base 205). In various embodiments, the indicator light 210 may be used to indicate the status of the solution distribution system 10. For example, the indicator light 210 may indicate the solution distribution system 10 is in operation or the solution distribution system 10 is offline. The indicator light 210 may provide such messages via different colors and/or patterns of lighting (e.g., flashing or solid).

In various embodiments, the power switch 215 includes a manual on/off switch. Alternatively, the power switch 215 may not include a manual on/off switch and as such may be powered on in an instance in which the power cord is attached to the power cord receiver of the power switch 215. The power cord receiver shape may be based on the desired power supplied to the solution distribution system 10. For example, the power receiver may be a standard power receiver used for similar voltages. The depicted electrical connection (e.g., the power receiver) is through a 120V/60 cy/1 phase grounded plug. The grounded plug may need a Ground Fault Interruption (GFI) outlet installed generally proximate to the enclosure body 135 (e.g., 6 feet in an instance in which a power cord 140 is 6 feet long) for receiving a power plug of the power cord 140.

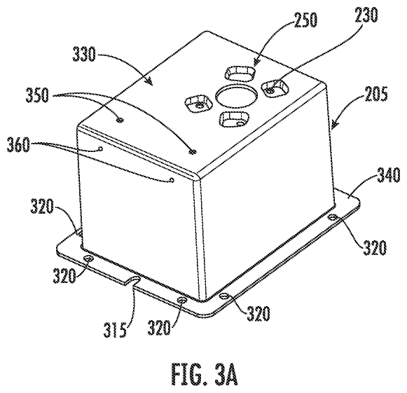

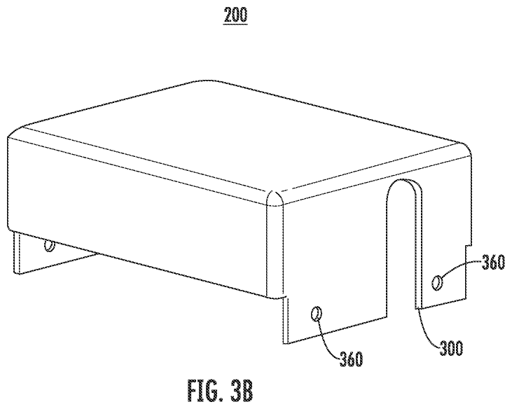

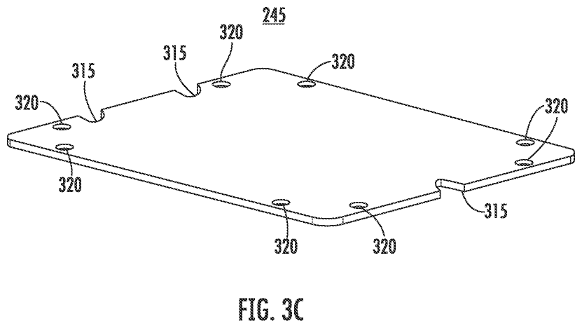

The depicted dispersion valve assembly 235 (e.g., three-way valve) is fluidly coupled to the delivery hose 115, the suction hose 120, and the bleed hose 125. The dispersion nozzle assembly 100 may be attached to the delivery hose 115 at an end opposite the dispersion valve assembly 235 (e.g., three-way valve). The fluid pickup assembly 155 may be attached to the suction hose 120 at an end opposite the dispersion valve assembly 235 (e.g., three-way valve). The delivery hose 115, the suction hose 120, and/or the bleed hose 125 may be removably connected to the dispersion valve assembly 235 (e.g., a three-way valve may have threading to receive a given hose). The depicted enclosure top plate 200 includes a plurality of apertures (shown in FIG. 3B) configured to receive each given hose 115, 120, 125 to pass therethrough.

Referring now to FIGS. 3A-3C, the enclosure base 205 (FIG. 3A), the enclosure top plate 200 (FIG. 3B), and the enclosure cover plate 245 (FIG. 3C) of an example embodiment are shown. In various embodiments, the enclosure base 205 (FIG. 3A), the enclosure top plate 200 (FIG. 3B), and the enclosure cover plate 245 (FIG. 3C) are attachable to one another to form the enclosure body 135 shown in FIG. 1A. In one embodiment, the enclosure top plate 200 is rotatably coupled via a hinge to the enclosure base 205.

Referring now to FIG. 3A, a perspective view of an example enclosure base 205 is shown. In various embodiments, the enclosure base 205 defines a base cavity (beneath the top surface 330 shown in FIG. 3A). The pump 230 and controller 220 may be disposed within the base cavity. The top surface 330 of the depicted enclosure base 205 includes one or more pump apertures 250 configured to allow communication between the pump 230 and the dispersion valve assembly 235 (e.g., three-way valve not shown). The pump 230 may define one or more input/output tubes that are structured to operatively couple the pump 230 to the dispersion valve assembly 235 as will be appreciated by one of ordinary skill in the art in view of this disclosure.

The top surface 330 of the depicted enclosure base 205 includes one or more dispersion valve assembly attachment points 350 (e.g., the dispersion valve assembly 235 may be attached to the top surface 330 of the enclosure base 205 at the dispersion valve assembly attachment points 350 via screws or other fasteners). One or more top plate attachment points 360 (e.g., small apertures) is defined by the depicted enclosure base 205 (e.g., a surface perpendicular to the top surface 330). The depicted enclosure top plate 200 is attached to the enclosure base via the top plate attachment points 360 (e.g., the enclosure top plate may be attached to the enclosure base 205 via screws positioned through the one or more top plate attachment points 360).

The depicted enclosure base 205 defines a flanged surface 340 extending at least partially around the lower edge of the enclosure base 205. The depicted flanged surface 340 includes one or more cover plate attachment points 320 (e.g., small apertures) and/or enclosure mountings 315. The depicted enclosure base 205 is attached to the enclosure cover plate 245 via the cover plate attachment point(s) 320.

While various embodiments are shown herein, the depicted cover plate attachment points 320, dispersion valve assembly attachment points 350, top plate attachment points 360, and the enclosure mountings 315 are defined as apertures for receiving screws or other fasteners. Various other attachment methods may be used (e.g., pins, staples, welds, seals, adhesives, and/or the like).

In various embodiments, one enclosure mounting 315 may be defined along one side of the flanged surface 340 (shown in FIG. 3A). Two other enclosure mountings 315 may be defined along the side opposite the first (e.g., the “top” of the enclosure body 135 may have a single enclosure mounting, while the “bottom” of the enclosure body 135 may have two enclosure mountings). While three enclosure mountings 315 are shown in various embodiments herein, some embodiments may include more or less enclosure mountings (e.g., based on the main enclosure weight or other design parameters that will be apparent to one of ordinary skill in the art). Additionally, while the enclosure mountings 315 are shown as apertures defined for receiving screws, other attachment means or fasteners may be used for securing the enclosure body 135.

The depicted enclosure base 205 is a unitary piece (e.g., stamped out of a single piece of metal). Alternatively, the enclosure base 205 may be formed from a plurality of pieces attached to one another (e.g., such as by welding). The enclosure base 205 may be made out of aluminum, steel, plastic, and/or the like. Each component of the enclosure body 135 discussed herein may use one or more different materials (e.g., the enclosure cover plate 245 (shown in FIG. 3C) may be made out of aluminum, while each of the enclosure base 205 (FIG. 3A) and the enclosure top plate 200 (FIG. 3B) may be made out of aluminum. In various embodiments, all of the components of the enclosure body 135 may be made out of aluminum.

Referring now to FIG. 3B, an example enclosure top plate 200 is shown. The depicted enclosure top plate 200 may define a bleed hose aperture 300, a delivery hose aperture (not shown), and/or a suction hose aperture (not shown). Each of the bleed hose aperture 300, the delivery hose aperture, and the suction hose aperture is defined on a different side of the depicted enclosure top plate 200 (e.g., each of the delivery hose aperture and the suction hose aperture are defined on a side not shown in FIG. 3B). In various embodiments, the position of the bleed hose aperture 300, the delivery hose aperture, and the suction hose aperture may be based on the design of the dispersion valve assembly 235 (e.g., the shape of an example three-way valve may define the positioning of the given aperture). In various embodiments, the size of each of the bleed hose aperture 300, the delivery hose aperture, and the suction hose aperture may be based on the size of the respective hose passing therethrough.

The depicted enclosure top plate 200 also includes one or more top plate attachment points 260 configured to allow the enclosure top plate 200 to be attached to the enclosure base 205. In various embodiments, other attachment mechanisms may be used in place of an aperture (e.g., a pin or clipping mechanism).

Referring now to FIG. 3C, the enclosure cover plate 245 is provided. The depicted enclosure cover plate 245 is a flat plate. The depicted enclosure cover plate 245 includes one or more cover plate attachment points 320 configured to attach the enclosure cover plate 245 to the enclosure base 205 (e.g., a screw through a cover plate attachment point 320 of the enclosure base 205 and through the corresponding cover plate attachment point 320 of the enclosure cover plate 245). The depicted enclosure cover plate 245 also defines one or more enclosure mountings 315 corresponding to the enclosure mounting of the enclosure base 205 (e.g., allowing the enclosure body 135 to be mounted and secured for operation).

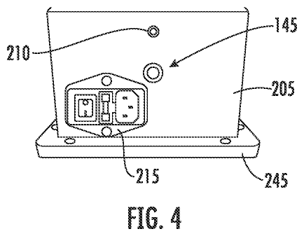

Referring to FIG. 4, an example enclosure base 205 is provided. The enclosure base 205 defines apertures to receive the indicator light 210, the power switch 215, and the purge button 145 as discussed above in reference to FIG. 2B. As shown, the power switch 215 may include an on/off switch and/or a power receiver.

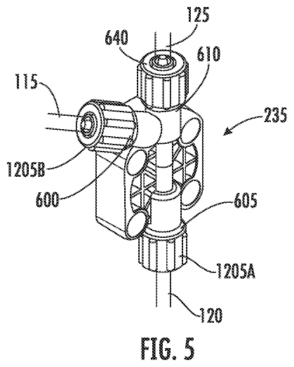

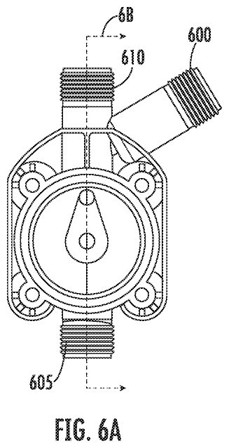

FIGS. 5-6B depict dispersion valve assemblies 235 of various embodiments. FIG. 5 shows an example dispersion valve assembly. FIG. 6A illustrates a top view of an example dispersion valve assembly. FIG. 6B is a cross-sectional view of the example dispersion valve assembly shown in FIG. 6A along cut line 6B.

The depicted dispersion valve assembly 235 of FIG. 5 is a three-way valve. The depicted dispersion valve assembly 235 is in communication with the pump 230 of the pump module, such that the solution is drawn from the fluid pickup assembly 155 to the dispersion nozzle assembly 100 via the pump 230. In various embodiments, the depicted dispersion valve assembly 235 is the control valve as discussed herein.

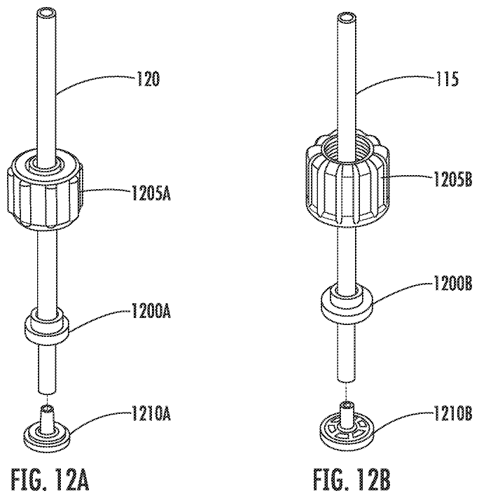

The depicted dispersion valve assembly 235 is connected to the delivery hose 115 via a delivery hose connector 600 (e.g., the delivery hose 115 may have a threading nut 1205B discussed in more detail in reference to FIG. 12B that removably connects to the delivery hose connector 600). The depicted delivery hose connector 600 is threaded. The depicted delivery hose 115 fluidly couples the dispersion valve assembly 235 with the dispersion nozzle assembly 100.

The depicted dispersion valve assembly 235 is connected to the suction hose 120 via a suction hose connector 605 (e.g., the suction hose may have a threading nut 1205A discussed in more detail in reference to FIG. 12A that removably connects to the suction hose connector 605). The depicted suction hose connector 605 is threaded. As discussed herein, the delivery hose 115 may fluidly couple the dispersion valve assembly 235 with the fluid pickup assembly 155. The solution flow path is defined from the fluid pickup assembly 155 to the dispersion nozzle assembly 100 through the dispersion valve assembly 235.

The depicted dispersion valve assembly 235 is connected to the bleed hose 125 via a bleed hose connector 610 (e.g., the bleed hose 125 may have a threading nut 640 that removably connects to the bleed hose connector 610). In other embodiments, other hose fastener arrangements may be used to connect the bleed hose 125, the suction hose 120, and the delivery hose 115 to the dispersion valve assembly 235 as may be apparent to one of ordinary skill in the art in view of this disclosure.

Referring to FIG. 6B, the dispersion valve assembly 235 defines a pathway between the suction hose connector 605 and either the delivery hose connector 600 or the bleed hose connector 610 at a given time. The dispersion valve assembly 235 may move between a delivery mode, a bleeding mode, and a non-operating mode. The delivery mode may be defined as the position or configuration of the dispersion valve assembly 235 that causes solution to be delivered to the dispersion nozzle assembly 100. The bleeding mode may be defined as the position or configuration of the dispersion valve assembly 235 that causes the system to be purged of solution (or air) via the bleed hose 125. The non-operating mode may be defined as the position or configuration of the dispersion valve assembly 235 that blocks any pathway to the delivery hose 115 or the bleed hose 125. The dispersion valve assembly 235 may be a mechanical valve configured to mechanically block the pathway to a given connector.

In various embodiments, during the dispersal of the solution, the dispersion valve assembly 235 is configured in the delivery mode. In one embodiment, the dispersion valve assembly 235 is moved into the delivery mode via the pump 230 and/or the controller 220. For example, the dispersion valve assembly 235 may be actuated to cause the pathway 630 between the suction hose connector 605 and the delivery hose connector 600 to be connected (e.g., completing the solution flow path discussed herein from the fluid pickup assembly 155 to the dispersion nozzle assembly 100). The dispersion valve assembly 235 may include one or more actuation mechanisms (not shown) that are structured to mechanically move between various positions to block one pathways to one or more given connectors. For example, the actuation mechanism may be a moveable pin (not shown) structured to block or unblock various connectors and thereby move the dispersion valve assembly 235 between the delivery mode, the bleeding mode, and the non-operating mode as will be apparent to one of ordinary skill in the art in view of this disclosure.

In various embodiments, once the dispersion event of the solution is complete (or before it has begun), the dispersion valve assembly 235 may be in the non-operating mode. As such, in non-operating mode, the connection pathway 630 between the suction hose connector 605, the delivery hose connector 600, and the bleed hose connector 610 may be blocked via the actuation mechanism, such that the solution is not provided to the dispersion nozzle assembly 100 or the bleed hose 125.

In some embodiments, during the non-operating mode, the connection pathway 630 between the suction hose connector 605 and the delivery hose connector 600 is not necessarily mechanically blocked. For example, in an instance in which the dispersion nozzle assembly 100 is mounted at a location higher than the pump module 2000, then the powering down of the pump 230 is sufficient to reduce the flow pressure of the solution and, as such stop the flow of the solution out of the dispersion nozzle 105.

In various embodiments, the dispersion valve assembly 235 (e.g., three-way valve) may be moved to bleeding mode in an instance the solution distribution system 10 is being purged. The example actuator mechanism is structured to connect the bleed hose connector 610 to the suction hose connector 605 (e.g., mechanically block the delivery hose connector 600 and/or unblocking the bleed hose connector 610). For example, the actuator mechanism of a three-way valve may mechanically move to allow the connection between the suction hose connector 605 and the bleed hose connector 610 (e.g., blocking any connection to the delivery hose connector 600).

The bleeding mode may be used to remove air and/or solution from the solution flow path (e.g., the bleeding mode may be engaged during the purge cycle). The length of time that the bleeding mode is engaged indicates the amount of air and/or solution removed from the solution flow path. The air is removed first and as the air is removed, solution begins to also flow into the bleed hose 125. The bleed hose 125 depicted in FIG. 2A is disposed within the solution container 150 at the end opposite the bleed hose connector 610, such that any solution removed from the solution flow path is returned to the solution container 150 (e.g., no solution is wasted during the purge process). The bleeding mode may be used to remove air from the solution flow path (e.g., a 6 to 20 second purge may remove any unwanted air from the solution flow path). Additionally or Alternatively, a longer purge cycle may allow for some or all of the solution to be removed from the solution flow path (e.g., a longer purge cycle may be used to remove any solution from the solution flow path for maintenance or the like).

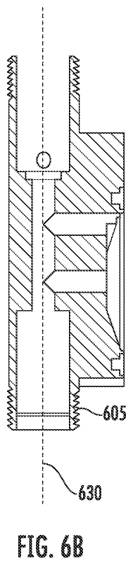

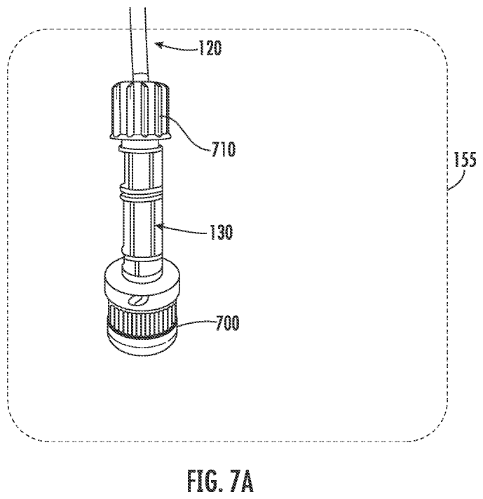

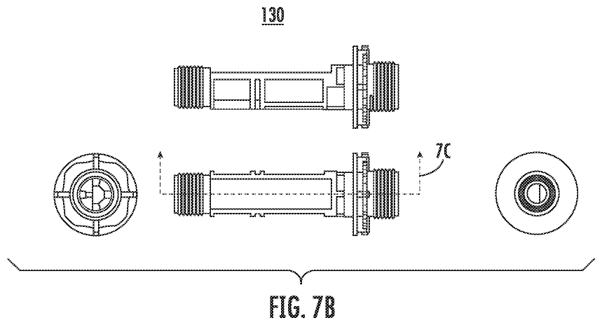

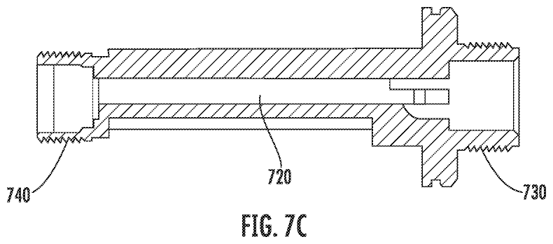

Referring now to FIGS. 7A, the fluid pickup assembly 155 of an example embodiment is shown. The depicted fluid pickup assembly 155 includes a pick-up valve 130 and a strainer module 700. The depicted fluid pickup assembly 155 is removably coupled to the suction hose 120 via a threading nut 710. Various other types of connections between the suction hose 120 and the fluid pickup assembly 155 may be envisioned.

The depicted pick-up valve 130 is a monolithic structure. The solution is configured to pass through the pick-up valve 130 upon activation of the pump module (e.g., via suction pressure created by the pump). In various embodiments, the solution may be provided to the pump module 2000 upon activation of the pump 230 (e.g., the solution may enter the pick-up valve due to suction pressure when the dispersion valve assembly 235 moves into the delivery mode that connects the suction hose 120 to the delivery hose 115). In various embodiments, upon installation, the fluid pickup assembly 155 may be installed below the pump module to allow for the solution to be carried through the pick-up valve 130 only due to suction pressure and without gravity playing a role to draw solution into the pick-up valve 130.

The depicted fluid pickup assembly 155 is weighted, such that the fluid pickup assembly 155 is structured to remain at the bottom of the solution container 150 during operation even given the buoyancy of the plastic or composite materials that might be used to manufacture the fluid pickup assembly 155.

In the depicted embodiment, the bottom (e.g., bottom plate or surface of strainer module 700) of the fluid pickup assembly 155 is weighted and the fluid pickup assembly 155 is manufactured by relatively buoyant polymer material(s). In this way, the fluid pickup assembly 155 is configured to rest on the bottom of the solution container 150 but remain in an upright position.

In various embodiments, the bottom of the fluid pickup assembly 155 may be at least 1 ounce. The weighted bottom portion of the fluid pickup assembly 155 may be lead. The bottom of the fluid pickup assembly 155 may define a twist-lock connection that is configured for coupling to the strainer module 700 of the fluid pickup assembly 155 as discussed below.

The depicted strainer module 700 defines a cylindrical filter body. The depicted strainer module 700 is a made from a polymer mesh, non-woven, or other filter material. The strainer module 700 is structured to limit sediment within the solution container from entering the solution flow path. The bottom surface or end of the strainer module 700 may be weighted as discussed above.

In various embodiments, the solution in the solution container 150 (shown in FIG. 2A) is drawn into the fluid pickup assembly 155 through the strainer module 700 and into the pick-up valve 130. The solution is carried through the pick-up valve 130 into the suction hose 120, into the dispersion valve assembly 235 and subsequently into the delivery hose 115, before entering the dispersion nozzle assembly 100 and exiting via the dispersion nozzle 105.

FIGS. 7B-7C depict a pick-up valve 130 of an example embodiment. FIG. 7B illustrates various orthogonal views of an example pick-up valve 130. FIG. 7C illustrates a cross-section view of the pick-up valve 130 shown in FIG. 7B along cut line 7C. The pick-up valve 130 may be made out of plastic or a similar material. The depicted pick-up valve 130 defines a fixed diameter (e.g., ¼ inch inner diameter plastic tubing may be used).

The pick-up valve 130 defines a flow channel 720 that, upon opening of the pick-up valve, allows the solution to move from the solution pickup inlet 730 to the suction hose pick-up connector 740. In various embodiments, once the solution enters the pick-up valve 130, the pump 230 maintains the pressure from approximately 50 PSI to approximately 55 PSI.

The depicted strainer module 700 is attached to the pick-up valve at the solution pickup inlet 730 (e.g., the solution moves through the strainer module 700 before entering the flow channel 720). The depicted solution pickup inlet 730 is threaded, such as to connect the strainer module 700. In some embodiments, the solution pickup inlet 730 may have other types of inlets besides the one provided in FIG. 7A. Additionally, the suction hose pick-up connector 740, may be threaded, such as to removably coupled with a threading nut 710. In various embodiments, the pick-up valve 130 and the suction hose 120 may be non-removably coupled (e.g., the suction hose 120 may be attached to the pick-up valve 130 via an adhesive or clamping device).

FIGS. 8A-8D are detailed component views of certain example components of the backflow pressure regulator 110 shown in FIG. 1A. FIG. 8A includes a bottom view of an example regulator base 800. FIG. 8B illustrates a cross-sectional view of the example regulator base 800 shown in FIG. 8A along cut line 8B. FIG. 8C illustrates various orthogonal views of an example regulator cover 830. FIG. 8D illustrates a cross-sectional view of the example regulator cover 830 shown in FIG. 8C along cut line 8D.

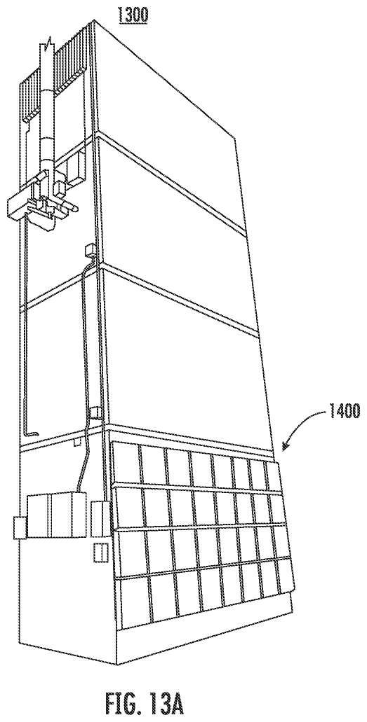

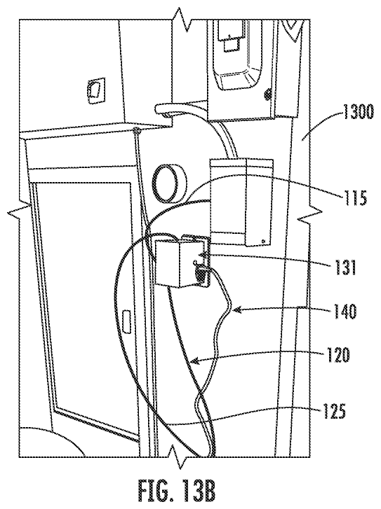

In various embodiments, the backflow pressure regulator 110 (shown in FIG. 1A) includes a regulator base 800 (FIGS. 8A-8B) and a regulator cover 830 (FIGS. 8C-8D). In various embodiments, the regulator base 800 may include one or more regulator attachment points 820A, 820B configured to mount the backflow pressure regulator 110 for operation (e.g., via screws or other fasteners inside of an air handling system, such as an air rotation unit shown in FIGS. 13A).

The regulator base 800 and/or the regulator cover 830 may be made of a plastic, a polyamide, and/or the like. For example, the regulator base 800 and the regulator cover 830 may be made using a polyamide powder filled with glass particles (PA-GF). Various other materials may be used to manufacture the regulator base 800 and the regulator cover 830. The backflow pressure regulator 110 may define multiple individual components (e.g., the regulator base 800 and the regulator cover 810) to allow for disassembly and improved accessibility to the backflow pressure regulator 110 (e.g., to allow for an operator to perform maintenance on the backflow pressure regulator 110).

As shown in FIG. 8A-8B, the depicted regulator base 800 is configured with a regulator delivery hose connector 810A and a regulator nozzle connector 810B. The depicted regulator delivery hose connector 810A is threaded (e.g., to receive a threading nut of the delivery hose 115). The depicted regulator delivery hose connector 810A is fluidly connected to the regulator inlet channel 890A. In an example embodiment, the solution may travel along the delivery hose 115 through the regulator delivery hose connector 810A and into the regulator cover 830 (shown in FIG. 8C). In the depicted embodiment, the regulator inlet channel 890A defines a right angle to divert the solution on its path to the regulator cover 830.

The depicted regulator nozzle connector 810B is threaded (e.g., to receive the dispersion nozzle 105). The depicted regulator nozzle connector 810B is fluidly connected to the regulator outlet channel 890B. In an example embodiment, the solution is configured to travel from the regulator cover 830 (shown in FIG. 8C) along the regulator outlet channel 890B and through the regulator nozzle connector 810B into the dispersion nozzle 105. The depicted regulator outlet channel 890B defines a right angle to divert the solution from the regulator cover 830 to the dispersion nozzle 105.

Referring now to FIGS. 8C-8D, the regulator cover 830 of an example embodiment is shown. The depicted regulator cover 830 defines a fluid cover aperture 831. The depicted fluid cover aperture 831 defines a threaded portion 845 extending at least partially along its length as shown. The depicted fluid cover aperture 831 defines a cover inlet 832 that is configured to attach to the regulator base 800.

The threaded portion 845 of the fluid cover aperture 831 is structured to receive a pressure adjustor 840. The depicted pressure adjustor 840 is a threaded screw or plug that is moveable along the threaded portion 845 of the fluid cover aperture 831 to change the pressure of the solution moving along a regulator pathway (e.g., via increasing or decreasing the volume of a cavity defined between the cover inlet 832 and the pressure adjustor 840). For example, as the pressure adjustor 840 moves towards the cover inlet 832 along the threaded portion 845, the pressure of the solution flowing along the regulator pathway may increase thereby producing an increased pressure of the solution at the dispersion nozzle 105. In such an example, as the pressure adjustor 840 moves away from the cover inlet 832 along the threaded portion 845, the pressure of the solution flowing through the regulator pathway may decrease thereby reducing the pressure of the solution at the dispersion nozzle 105.

The depicted pressure adjustor 840 defines an adjustment feature that is configured for user engagement during an adjustment operation. For example, as shown in FIGS. 8E, the pressure adjustor 840 may define a flat interface cavity that is sized to receipt a flathead screwdriver for rotating clockwise or counter-clockwise during adjustment operations. In other embodiments, the adjustment feature may be a knob or handle (not shown) that is configured for direct user engagement.

FIG. 8E depicts an assembled dispersion nozzle assembly 100 comprising a backflow pressure regulator 110 and a dispersion nozzle 105. The depicted backflow pressure regulator 110 is connected to a delivery hose 115, that provides the solution to the dispersion nozzle assembly 100. The backflow pressure regulator 110 includes the pressure adjustor 840 discussed above in reference to FIGS. 8C-8D.

The depicted backflow pressure regulator 110 is structured to be adjusted in order to maintain a sufficient pressure at the dispersion nozzle 105. An operator or user may move the pressure adjustor 840 in order to change the pressure of the solution leaving the dispersion nozzle 105 (e.g., the pressure adjustor 840 may be adjusted in an instance the flow of the solution out of the dispersion nozzle 105 is determined as either too strong or too weak). During typical operations, the pressure adjustor 840 remains in a fixed position, such that a relatively stable pressure for the solution is maintained. In an instance in which the depicted pressure adjustor 840 is turned in the clockwise direction, the pressure of the solution travelling within backflow pressure regulator 110 is increased (e.g., the pressure adjustor 840 moves towards the cover inlet 832 shown in FIG. 8D). Alternatively, in an instance in which the depicted pressure adjustor 840 is turned in the counter-clockwise direction, the pressure of the solution travelling within the backflow pressure regulator 110 is decreased (e.g., the pressure adjustor 840 moves away from the cover inlet 832 shown in FIG. 8D).

The depicted dispersion nozzle 105 is attached to the backflow pressure regulator 110 via a threading nut (e.g., the dispersion nozzle head 105A may be attached to an assembly configured to be attached to the backflow pressure regulator 110). The depicted dispersion nozzle 105 is attached to the backflow pressure regulator 110 opposite the attachment of the delivery hose 115.

The backflow pressure regulator 110 may be adjusted to ensure the proper dispersion pressure at the dispersion nozzle 105. For example, the dispersion pressure at the dispersion nozzle 105 may be defined from approximately 50 pounds per square inches (PSI) to approximately 55 PSI. In some embodiments, such as those where the solution flow path defines a relatively long run (e.g., the dispersion nozzle assembly 100 is mounted at least 15 feet or more from the pump enclosure assembly 131), the dispersion pressure may be set higher pressures. For example, in such circumstances, the dispersion pressure may be set at 101.5 PSI rather than 55 PSI.

The depicted dispersion nozzle assembly 100 is structured to be mounted within an air handling system. For example, the dispersion nozzle assembly 100 may be mounted within the air handling system, while the enclosure body 135 may be mounted remote from the dispersion nozzle assembly 100 (e.g., the enclosure body 135 may be mounted on the exterior of the air handling system). The air handling system may be an air rotation unit, such as the air rotation unit 1300 shown in FIGS. 13A-13B. The dispersion nozzle assembly 100 may be structured to be mounted above the pump module 2000 to allow for the solution to remain in the solution flow pathway during non-operation (e.g., in an instance in which the pump 230 is turned off, the pressure of the solution is not sufficient to overcome the force of gravity and the solution will remain within the solution flow pathway).

The depicted dispersion nozzle assembly 100 may be designed to be disposed upstream of a heating and/or cooling coil of an HVAC system. As such, the solution being dispersed via the dispersion nozzle 105 may enter the central core area of the air stream travelling along the HVAC system. In various embodiments, the positioning of the dispersion nozzle assembly 100 may encourage the dispersed fluid particles in the solution to remain airborne and are less likely to contact and remain trapped on duct walls (e.g., by entering the air steam and leaving the air handling system).

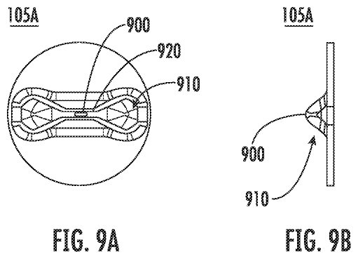

FIGS. 9A and 9B depicts three orthogonal views of a dispersion nozzle head 105A structured in accordance with one example embodiment. The depicted dispersion nozzle head 105A includes a generally flat body portion made out of stainless steel that defines a curvilinear shaping flange 910 extending therefrom. In some embodiments, the curvilinear shaping flange is formed into the body portion of the dispersion nozzle head 105A by a stamping operation.

The depicted dispersion nozzle head 105A defines a nozzle outlet 900 positioned within its curvilinear shaping flange 910. The depicted dispersion nozzle head 105A defines a singular nozzle outlet 900 but other embodiments might have two or more nozzle outlets (not shown). The depicted curvilinear shaping flange 910 defines a narrow portion 920 proximate the nozzle outlet 900 (e.g., creating an hourglass type shape).

The depicted nozzle outlet 900 is structured, with the depicted curvilinear shaping flange 910, to dispense the anti-pathogenic solution into the air stream in a dispersion pattern. The dispersion pattern produced by the depicted dispersion nozzle head 105A is a flat fan dispersion pattern. The flat fan dispersion pattern is produced as solution exits the nozzle outlet 900 and is forced through the hourglass shape of the curvilinear shaping flange 910. In one embodiment, the flat fan pattern produced by the depicted dispersion nozzle head 105A may be approximately 12 inches to approximately 18 inches wide.

The dispersion pattern of the depicted dispersion nozzle 105 can be adjusted via the pressure adjustor 840 of the backflow pressure regulator 110 (shown in FIG. 8E). Upon initial startup, the solution potentially exits the dispersion nozzle head 105A in an undesired dispersion pattern (e.g., either too high of a dispersion pressure or too low of a dispersion pressure). In such an instance, the backflow pressure regulator is adjusted to reach the desired dispersion pattern (e.g., the backflow pressure regulator may be adjusted to form a fan shaped pattern). For example, in an instance in which the initial dispersion pressure has too little of a dispersion pressure, the pressure adjustor 840 of the backflow pressure regulator 110 can be turned in the clockwise direction to increase the dispersion pressure. Upon achieving the desired dispersion pattern, the pressure adjustor 840 of the backflow pressure regulator 110 remains in place to maintain the dispersion pattern for subsequent dispersion events.

The depicted dispersion nozzle head 105A is structured to deliver a dispersion event dosage of solution over a given dispersion event period. The dispersion event dosage may be from approximately 1.2 fluid ounces to approximately 1.5 fluid ounces of solution. The dispersion event period may be approximately 5 seconds to approximately 10 seconds. The dispersion event period may be from approximately 6 seconds to approximately 8 seconds. The dispersion nozzle head 105A may be structured to deliver from 1.2 fluid ounces to 1.5 fluid ounces of the solution over a 6 second dispersion event period.

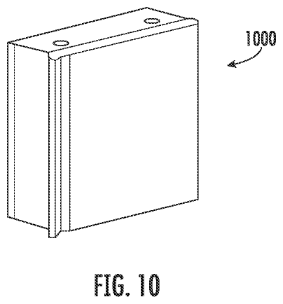

Referring now to FIG. 10, an example circuit board enclosure 1000 is provided. The depicted circuit board enclosure 1000 is configured as a box or housing to protect a circuit board (e.g., controller 220 shown in FIG. 11) used to control the dispersion of the solution. In various embodiments, the circuit board enclosure 1000 may be made out of a plastic, a polycarbonate, stamped metal, and/or a similar material. The circuit board enclosure 1000 may be mounted within the enclosure body 135 discussed herein.

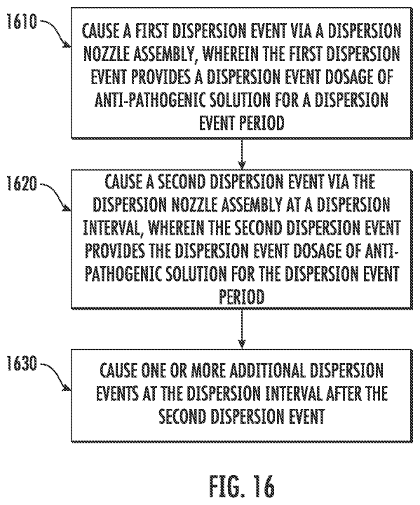

FIG. 11 is a schematic illustration of a controller 220 structured in accordance with embodiments of the present invention. The depicted controller 220 includes a printed circuit board (PCB) 1100. The PCB 1100 includes one or more processors, non-transitory memory, and/or the like. The PCB 1100 is configured to execute the computer program instructions discussed below in reference to FIG. 16.

In various embodiments, the PCB 1100 includes means for actuating the dispersion valve assembly 235 and/or the pick-up valve 130. The PCB 1100 also may include means for operating the solution distribution system 10 to provide a specific dispersion event period and dispersion pressure. The PCB 1100 may also include means for activating and deactivating the solution distribution system 10 at a dispersion interval (e.g., by activating and deactivating the pump 230 and the dispersion valve assembly 235).

The PCB 1100 may also include means for causing a first dispersion event via the dispersion nozzle assembly. The PCB 1100 may also include means for causing a second dispersion event via the dispersion nozzle assembly. The PCB 1100 may also include means for monitoring one or more pathogens that may be present in an air stream and for engaging the control valve (e.g., the dispersion valve assembly 235) to dispense anti-pathogenic solution into the monitored air stream when the amount of detected pathogens exceeds a predetermined threshold.

The PCB 1100 may include, be associated with, or may otherwise be in communication with a processing circuitry, which includes a processor and a memory device, and a communication interface. In some embodiments, the processor (and/or co-processors or any other processing circuitry assisting or otherwise associated with the processor) may be in communication with the memory device via a bus for passing information among components of the apparatus. The memory device may be non-transitory and may include, for example, one or more volatile and/or non-volatile memories. In other words, for example, the memory device may be an electronic storage device (for example, a computer readable storage medium) comprising gates configured to store data (for example, bits) that may be retrievable by a machine (for example, a computing device like the processor). The memory device may be configured to store information, data, content, applications, instructions, or the like for enabling the apparatus to carry out various functions in accordance with an example embodiment of the present invention. For example, the memory device could be configured to buffer input data for processing by the processor. Additionally or alternatively, the memory device could be configured to store instructions for execution by the processor.

The processor of the PCB 1100 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor of the PCB 1100 may be configured to execute instructions stored in the memory device of the PCB 1100 or otherwise accessible to the processor. Alternatively or additionally, the processor may be configured to execute hard coded functionality. As such, whether configured by hardware or software methods, or by a combination thereof, the processor may represent an entity (for example, physically embodied in circuitry) capable of performing operations according to an embodiment of the present disclosure while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (for example, the computing device) configured to employ an embodiment of the present invention by further configuration of the processor by instructions for performing the algorithms and/or operations described herein. The processor 14 may include, among other things, a clock, an arithmetic logic unit (ALU) and logic gates configured to support operation of the processor.

As shown in FIG. 11, the PCB 1100 is electrically connected to the pump 230, the indicator light 210, the purge button 145, and the power switch 215. In various embodiments, the PCB 1100 may be powered via the power switch 215 (e.g., the power cord 140 may be plugged into the power switch 215). The PCB 1100 may provide power to the pump 230. In some embodiments, the PCB 1100 may also activate the indicator light 210. The PCB 1100 may receive a signal from the purge button 145 in an instance the purge button 145 has been engaged.

The controller 220 may be capable of adjusting the dispersion event period, the dispersion interval, and/or the dispersion pressure. In various embodiments, the controller 220 may have a plurality of settings that allows for the dispersion event period, the dispersion interval, and/or the dispersion pressure to be changed. Alternatively, the controller may be pre-programmed, such as by a manufacturer with specific dispersion event period, dispersion interval, and/or dispersion pressure.

FIG. 12A depicts a suction hose assembly of an example embodiment. The depicted suction hose assembly includes a suction hose 120, a threading nut 1205A, a holding ring 1200A, and a pipe holder 1210A. In some embodiments, the suction hose assembly may include a plurality of threading nuts 1205A provided a various points along its length (e.g., in some instances, one threading nut 1205A may be provided to removably couple with the pick-up valve 130 and another threading nut 1205A may be provided to removably couple with the dispersion valve assembly 235).

The depicted holding ring 1200A may be received by the threading nut 1205A to ensure a fluid-tight connection for the threading nut 1205A. The pipe holder 1210A may be inserted in the end of the suction hose 120 to provide additional rigidity to the suction hose 120 (e.g., the pipe holder 1210A may have a through-hole that allows the solution to pass therethrough). The depicted suction hose 120 is a flexible tubing (e.g., made out of plastic or the like).

FIG. 12B depicts a delivery hose assembly of an example embodiment. The depicted delivery hose assembly includes a delivery hose 115, a threading nut 1205B, a holding ring 1200B, and a pipe holder 1210B. The delivery hose assembly may include a plurality of the threading nuts 1205B extending along its length (e.g., in some instances, one threading nut 1205B may be provided to removably couple with the backflow pressure regulator 110 and another threading nut 1205B may be provided to removably couple with the dispersion valve assembly 235). In such multiple threading nut examples, additional holding rings 1200B and pipe holders 1210B may be provided.

The depicted holding ring 1200B may be received by the threading nut to ensure a fluid-tight connection. The pipe holder 1210B may be inserted in the end of the delivery hose 115 to provide additional rigidity to the delivery hose 115 (e.g., the pipe holder 1210B may have a through-hole that allows the solution to pass therethrough). The depicted delivery hose 115 is formed from a flexible tubing (e.g., made out of plastic or the like).

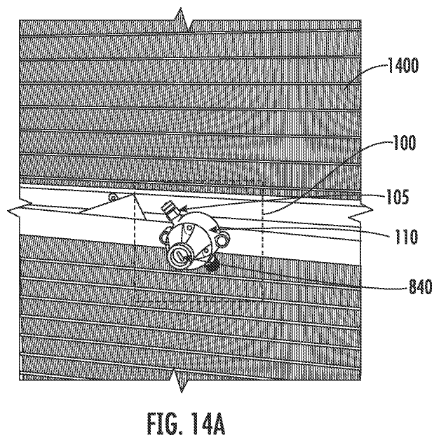

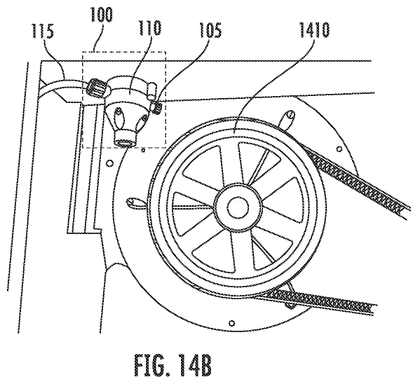

Referring now to FIGS. 13A-13B, an example air handling system (e.g., air rotation unit 1300) is provided. FIGS. 13B, 14A, and 14B show various components of an example solution distribution system 10 of various embodiments installed on an air rotation unit 1300. While various examples discussed herein are described in reference to an air rotation unit 1300, various embodiments of the solution distribution system 10 may be used with various other air handling systems as will be apparent to one of ordinary skill in the art.

In some embodiments, a selected air rotation unit 1300 may be used in conjunction with one or more additional air rotation units (not shown). In such circumstances, a solution distribution system 10 may be provided for one or more of said air rotation units. For example, a single solution distribution system 10 may be configured to treat an environment managed by several air rotation units. In other embodiments, each air handling system (e.g., air rotation unit 1300) may receive its own installed solution distribution system 10. As will be appreciated by one of ordinary skill in the art, air handling systems such as the depicted air rotation unit 1300 include one or more heating and/or cooling coils 1400 as discussed in reference to FIG. 13C.

As shown in FIG. 13B, the depicted pump enclosure assembly 131 is mounted on the exterior of the air rotation unit 1300. The depicted pump enclosure assembly 131 is mounted on the air rotation unit 1300 via screws or other fasteners. The solution container 150 (not shown in FIG. 13B) may also be disposed on the exterior of the air rotation unit 1300. In various embodiments, the solution container may be mounted below the enclosure body 135 to allow for proper operation. As shown, the delivery hose 115 may enter the interior of the air rotation unit 1300 (e.g., via a hole in the air rotation unit) in order to connect to the dispersion nozzle assembly 100 mounted inside the air rotation unit 1300 (as shown in FIG. 14A). The pump enclosure assembly 131 may also be mounted within the air handling system (e.g., within the air rotation unit 1300). The pump enclosure assembly 131 may be mounted at any non-freezing location internal or external to the air handling system (e.g., air rotation unit 1300).

The depicted enclosure body 135 includes the controller 220 and the pump module (e.g., the pump 230 and the dispersion valve assembly 235). The depicted pump enclosure assembly 131 (e.g., the enclosure body 135 and internal components) defines a main enclosure weight. The main enclosure weight may be less than 5 kilograms (e.g., to allow for simpler installation).

Referring now to FIG. 14A, the dispersion nozzle assembly 100 of an example embodiment is shown installed within an air rotation unit 1300. The dispersion nozzle assembly 100 is configured to be mounted in a stable and accessible location within a given air handling system.

The dispersion nozzle assembly 100 may be structured to be positioned to discharge solution onto a hard surface of the air handling system. The hard surface in which the dispersion nozzle assembly 100 disperses the solution onto may be any surface in which an air stream passes over (e.g., to allow the solution to enter the air stream. The hard surface that receives the dispersed solution may be the coil 1400 (as shown in FIG. 14A) or a fan (as shown in FIG. 14B). Additionally or alternatively, the hard surface may be part of the frame of the air handling system (e.g., the cabinetry framework or squirrel cage of the air rotation unit 1300). Various other surfaces may be contemplated as long as the given surface is positioned within or along the air stream path of the air handling system.

The depicted dispersion nozzle assembly 100 is mounted near the return air port of the air handling system. The dispersion nozzle assembly 100 may be mounted upstream of a heating and/or cooling coil of the air rotation unit. For example, the depicted dispersion nozzle 105 is directed towards the coil 1400 shown in FIG. 14A. The dispersion nozzle 105 may be directed, when mounted, such that the solution dispersed enters the central core area of the air stream. The dispersion nozzle assembly 100 may be attached via screws and/or other attachment methods (e.g., the dispersion nozzle assembly 100 may be screwed to the air rotation unit via the one or more regulator attachment points 820A, 820B on the regulator base 800 of the backflow pressure regulator 110.

Referring now to FIG. 14B, another example mounting location within an air rotation unit for a dispersion nozzle assembly 100 is shown. In various embodiments, the dispersion nozzle assembly 100 may be structured to be mounted with the dispersion nozzle 105 positioned facing the supply fan 1410. For example, the dispersion nozzle assembly 100 may be installed at a location such that the dispersion nozzle 105 directs the solution into the supply fan 1410 for distribution. FIG. 14B illustrates a horizontal air handling system (e.g., the solution distribution system 10 may be implemented in various sized air handling systems).

### Method of Assembling a Solution Distribution System

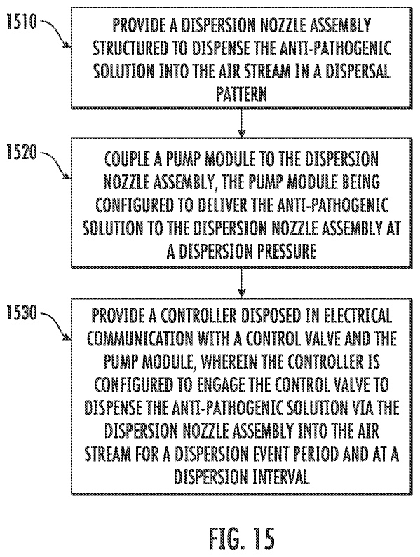

Referring now to FIG. 15, a method of assembling a solution distribution system, such as various embodiments discussed herein, is provided. The solution distribution system 10 may be configured to dispense an anti-pathogenic solution into an air stream managed by an air handling system. As discussed above in reference to FIG. 13A, the air handling system may be an air rotation unit 1300. In various embodiments, the air handling system may be a draw through or blow through air handler.

The anti-pathogenic solution may, in various embodiments, be non-metallic. The solution may have a pH value of between approximately 1.5 and approximately 1.6. The solution may have a specific gravity of between approximately 1.10 and approximately 1.30.

Referring now to Block 1510 of FIG. 15, the method of assembly a solution distribution system 10 may include providing a dispersion nozzle assembly 100 structured to dispense the anti-pathogenic solution into the air stream in a dispersion pattern. As discussed above in more detail in reference to FIGS. 8A-8E, the dispersion nozzle assembly 100 may include a backflow pressure regulator 110. As discussed below in reference to Block 1520 and 1530, the backflow pressure regulator 110 may be disposed along the solution flow path between the control valve (e.g., the dispersion valve assembly 235 or the pick-up valve 130) and the dispersion nozzle 105. The backflow pressure regulator 110 may be structured to limit air introduction into the solution flow path.

The dispersion nozzle 105 may be structured to produce an angular fan dispersion pattern of solution. The dispersion pattern of solution may be a pulsating spray. The pulsating spray may have a pulsation period that is a subset of the dispersion event period. The pulsating spray may be defined as a 0.5 second burst for a total dispersion event period of 6 seconds (12 pulses during the dispersion event period).

The dispersion nozzle 105 may be structured to distribute the solution in the angular fan dispersion pattern at a desired molecule size. For example, the dispersion nozzle 105 may be structured to distribute the solution in the angular fan dispersion pattern at a molecule size of between approximately 8 and approximately 15 microns. In various embodiments, the size of the molecule may assist the distribution of the solution into the air stream. The dispersion nozzle 105 may be structured for mounting upstream of a heating or cooling coil of the air rotation unit (e.g., as shown in FIG. 14A).

In some embodiments, the dispersion nozzle 105 may be structured to disperse the anti-pathogenic solution as formulated to pass a Virucidal Hard-Surface Efficacy Test for Severe Acute Respiratory Syndrome-related Coronavirus 2 (SARS-CoV-2)(COVID-19 Virus) under Protocol No. GLO.1.07.01.20.

Referring now to Block 1520 of FIG. 15, the method of assembly a solution distribution system 10 may include coupling a pump module to the dispersion nozzle assembly. The pump module may be configured to deliver the anti-pathogenic solution to the dispersion nozzle assembly 100 at a dispersion pressure. The pump module may be coupled to the dispersion nozzle assembly 100 via a delivery hose 115.

The pump module 2000 may include a pump 230 and a dispersion valve assembly 235. The pump 230 may be configured to draw the solution from the pick-up valve 130 and provide said solution to the dispersion nozzle assembly 100. The controller 220 and the pump module may be the only draw of electricity by the solution distribution system 10 during operation (e.g., the solution distribution system 10 may draw approximately 0.8 amps during a dispersion event). The method may also include enclosing the pump module and the controller within the enclosure body 135. The pump enclosure assembly 131 (e.g., the enclosure body, the pump module, and the controller 220) may define a main enclosure weight. The main enclosure weight may be less than 5 kilograms. The enclosure body 135 may be configured for mounting to an exterior wall of the air rotation unit (e.g., as shown in FIG. 13B). The pump module may allow for free flow of the liquid without air entrainment.

In various embodiments, the method of assembling a solution distribution system 10 may also include providing a fluid pickup assembly 155 including a pick-up valve 130 coupled to the pump module. The fluid pickup assembly 155 (and subsequently the pick-up valve 130) may be coupled to the pump module via the suction hose 120. The fluid pickup assembly 155 may also include a strainer module 700. The strainer module 700 may define a cylindrical filter body structured to limit sediment within the solution container from entering the solution flow path. The fluid pickup assembly 155 may be configured to be disposed within a solution container 150, such that the fluid pickup assembly 155 draws the solution from the solution container 150 into the solution flow pathway. The depicted strainer module 700 is weighted to remain upright within the solution container 150.

In various embodiments, the solution flow path may be defined between the pick-up valve 130 and the dispersion nozzle assembly 100. The solution flow path may begin at the pick-up valve 130 as the solution is drawn through the suction hose 120 via the pump, into the dispersion valve assembly 235 (e.g., three-way valve) and into the delivery hose 115 before ending up in the dispersion nozzle assembly 100.

Referring now to Block 1530 of FIG. 15, the method of assembling a solution distribution system 10 may include providing a controller 220 disposed in electrical communication with a control valve and the pump module. The controller 220 may be configured to engage the control valve to dispense the anti-pathogenic solution via the dispersion nozzle assembly 100 into the air stream for a dispersion event period and at a dispersion interval.

In various embodiments, the controller 220 may programmable (e.g., to allow the dispersion event period and/or dispersion interval to be changed). In various embodiments, the control valve may be the pick-up valve. Alternatively, the control valve may be the dispersion valve assembly 235 (e.g., three-way valve as actuated by the pump 230). The control valve may be actuated by the controller to move the solution distribution system 10 between a delivery mode, a bleeding mode, and a non-operating mode, as discussed above in reference to FIG. 6A. The actuation of the control valve may be based on the activation of the pump 230.

The dispersion event dosage may be based on the dispersion event period and the dispersion pressure of the solution distribution system 10. At least one of the dispersion event period or the dispersion pressure may be selected based on the desired dispersion event dosage of the solution. The desired dispersion event dosage of the solution may be between approximately 1.2 fluid ounces and approximately 1.5 fluid ounces into the air stream. The dispersion event period may be from approximately 6 seconds to approximately 8 seconds. Various other dispersion event periods may be used for other applications of the solution distribution system 10 (e.g., a residential unit may have a shorter dispersion event period). The dispersion pressure may be between approximately 50 PSI and approximately 55 PSI. The dispersion event dosage may be between approximately 1.2 fluid ounces and approximately 1.5 fluid ounces into the air stream in an instance in which the dispersion event period is approximately 6 seconds to approximately 8 seconds and the dispersion pressure is between approximately 50 PSI and approximately 55 PSI.

The dispersion interval may be the amount of time between each dispersion event. For example, the dispersion interval may be approximately 30 minutes to approximately 90 minutes. The dispersion interval may be approximately 60 minutes. In an example embodiment, the solution distribution system 10 may be configured to dispense approximately 1.2 fluid ounces and approximately 1.5 fluid ounces of the solution in 6 seconds, before waiting a dispersion interval of 60 minutes to dispense additional solution.

The dispersion event period, the angular fan dispersion pattern, and/or the dispersion event interval may be selected based on the number of solution molecules to be distributed to the air steam over a treatment period. For example, the dispersion event period, the angular fan dispersion pattern, and/or the dispersion event interval may be selected to distribute over ten million anti-pathogenic solution molecules into the air stream over a treatment period of between approximately 6 and approximately 8 hours. The anti-pathogenic solution of an example embodiment is distributed into the air stream by being impinged on one or more hard surfaces of the interior HVAC system (e.g., cabinet, coil(s), drain pan, fan, and/or blower assembly).

Method of Dispensing the Solution into an Air Stream

Referring now to FIG. 16, a method of dispensing an anti-pathogenic solution into an air stream managed by an air handling system is provided.

The solution distribution system of various embodiments is configured to produce dispersion events that are well suited for distribution into an air stream based on principles of Brownian motion to remove pathogens from the air. Brownian motion is the random movement of particles in a fluid medium (e.g., an air stream) due to their collisions with other atoms or molecules in the surrounding medium. Even though a particle may be large compared to the size of atoms and molecules in the surrounding medium, it can be set in motion by bombardment with many tiny, fast-moving masses. Brownian motion may be considered a macroscopic (visible) picture of a particle influenced by many microscopic random effects.

Because the movements of atoms and molecules in a liquid and gas is random, over time, larger particles will disperse evenly throughout the medium. If there are two adjacent regions of matter and region A contains twice as many particles as region B, the probability that a particle will leave region A to enter region B is twice as high as the probability a particle will leave region B to enter A. Diffusion, the movement of particles from a region of higher to lower concentration, can be considered a macroscopic example of Brownian motion.

Any factor that affects the movement of particles in a fluid impacts the rate of Brownian motion. For example, increased temperature, increased number of particles, small particle size, and low viscosity increase the rate of motion. Random movement and collision with other particles are highly affected by air velocity and space limitations. Thus, pathogenic particles (e.g., COVID-19 (SARS CoV2)) randomly moving in a confined spatial environment have an extremely high probability of coming in contact with a potentially lethal counter particle.

Embodiments of the solution distribution system are configured to dispense anti-pathogenic solution particles into an air stream that is managed by an air handling system in a manner designed to take advantage of Brownian motion principles. Each dispersion event produced by embodiments of the solution distribution system discussed herein includes anti-pathogenic solution that is micro vaporized into molecules ranging from 8 microns to 15 microns in size. The solution distribution system was designed to take advantage of the fact that virus-laden small (<5 μm) aerosolized droplets can remain in the air and travel long distances allowing for the potential aerosol transmission of COVID-19 (SARS-CoV-2). Various embodiments of the herein disclosed solution distribution system have an average dispersion rate of 5 gallons/18.927 liters per 3-week period. The total dispersion is not critically tied to a heating, ventilation, and air conditioning (HVAC) system total cubic feet per minute (cfm) (volume of air) as some other competing systems might require, but rather to the fact that the total number of cubic feet of molecular volume can reach 10 million individual solution molecules per 1 cubic meter of conditioned space circulating per hour.

Embodiments of the present disclosure provide introduction of small amounts of anti-pathogenic solution over a constant period of time (the very essence of toxicity: dose x duration). Since fresh air makeup will introduce additional pathogens there is a constant reintroduction of fresh anti-pathogenic solution to address both those recirculating airborne pathogens and provide continued surface protection on a daily basis. Surface protection is achieved through the addition of a non-chemical binding agent to the anti-pathogenic solution which allows some of the circulating molecules to adhere to the surface of an associated air handling system and to any surfaces that might be contacted by a treated air stream.

In order for the anti-pathogenic solution to attack and neutralize any pathogen, physical contact must be made between the solution and the pathogen. Physical contact with solution with the COVID-19-(SARS CoV2) results in disruption of the viral envelope membrane structure thereby nullifying the pathogenicity of the virus. Solution distribution systems as discussed herein are designed to introduce dispersion events of a defined size and frequency that introduce an extraordinary volume of anti-pathogenic molecules into a treated air stream that are toxic to COVID-19-(SARS CoV2). The air stream is circulated by an air handling system and the anti-pathogenic molecules remain in proximity to the virus ensuring that molecules of the solution bombard and thereby destroy the virus consistent with principles of Brownian motion.

Lethality (toxicity), as previously discussed, is a simple function of Dose x Duration. By providing constant anti-pathogenic solution in a manner that will allow impingement with the invading pathogen, positive results will be achieved. Embodiments of the present disclosure are designed to infuse a small amount of solution into the air stream on a continually timed basis. Frequency and amount of product infusion is based on application requirements.

Solution distribution systems that are structured as discussed herein are configured to consistently maintain sufficient quantities of properly sized anti-pathogenic solution molecules in an air stream to produce high levels of treatment efficacy and viral load reduction. The answer to providing long-term protection is attacking the problem to mitigate the ability of pathogens to remain airborne and recirculate over and over within the conditioned space without their coming in contact with the anti- pathogenic product and being neutralized. The solution distribution system was designed and developed to produce small micron product vaporization which would be entrained with the same air carrying the pathogens continuously throughout the building environmental system. This puts the problem (i.e., the pathogen) in proximity to the solution (i.e., properly sized and dispersed anti-pathogenic solution molecules).

Since the retention rate in moving air is actually logarithmic or exponential, the dwell time for the pathogen in moving air could be as long as 3 to 4 hours. That is indicative of the pathogens not only travelling within the solution distribution system but coming in contact with the entire surface area of that solution distribution system. This single factor is the key to permanent protection in buildings. Without physically having a Covid-19 disinfectant infused into the conditioned space all the filter will do is to pull the air with all the virus in it into the filter. That will cause the supply and return air filters to be infected with the virus. This will necessitate the installation of UV lights at the filters to kill those viruses. If the HVAC blowers are off and people come into the building, they will spread the virus. The HVAC blower will then have to run continuously all the time and even then, some of the virus will come into contact with surfaces in the building as they travel back into the return filter duct. You would need even more UV lights to disinfect those surfaces in the building. The investment for such schemes will be tremendous. The solution distribution system discussed herein allows for elimination of pathogens without the need for UV lights or additional safety features.

The method of FIG. 16 may be carried out by various embodiments of the solution distribution systems 10 discussed herein. In various embodiments, the solution distribution system 10 may include a computer program product that includes at least one non-transitory computer-readable storage medium having computer-executable program code portions stored therein. The computer-executable program code portions may include program code instructions. The computer executable program code portions may be carried out by the controller 220. The computer-executable program code portions may include program code instructions configured to carry out the methods discussed in reference to FIG. 16.

Referring now to Block 1610 of FIG. 16, the method of dispensing an anti-pathogenic solution into an air stream managed by an air handling system may include causing a first dispersion event via a dispersion nozzle assembly. The first dispersion event may provide a dispersion event dosage of anti-pathogenic solution for a dispersion event period. The first dispersion event and any subsequent dispersion events may provide the solution at a dispersion pressure (e.g., a dispersion pressure may be between 50 PSI and 55 PSI). The first dispersion event may be caused via any embodiment of the dispersion nozzle assembly 100 discussed herein. The solution may be dispensed upstream of the heating or cooling coil of the air rotation unit.

In various embodiments, the air volume in an area in which the air rotation unit is servicing may be monitored. For example, the air in a room being serviced by the air rotation unit may be monitored for one or more pathogens. The one or more pathogens tested for may be a microorganism, such as Staphylococcus aureus, Pseudomonas aeruginosa, Salmonella enterica, and/or various spores (e.g., Ascospores, Basidiospores, Bipolaris, Botrytis, Chaetomium, Cladosporium, Curvularia, Epicoccum, Fusarium, Myrothecium, Nigrospora, Penicillium, Pithomyces, Rusts, smuts, Periconia, Myxomycetes, Stachybotrys, Stemphylium, Torula, Ulocladium, Zygomycetes, pollen, skin cells, background debris, and/or hyphal fragments). The solution distribution system 10 may be structured to kill one or more pathogens discussed above. The solution distribution system 10 may be capable of killing various pathogens, including SARS-CoV-2 (COVID-19 Virus). The pathogens killed by the solution distribution system 10 may be based on the solution used in the solution distribution system 10. The monitoring of the air may be performed via a sensor. For example, a particle counter sensor can be used to monitor particle size (e.g., the particle counter sensor can be configured to identify one or more fungi, bacteria, yeast, and/or virus based on a certain particle size). The monitoring may be automatic or manually (e.g., a user may check the air quality with a handheld sensor). The monitoring may be continuous (e.g., a sensor may be installed to continuously monitor the air quality). Alternatively, the monitoring may be periodic at a predetermined monitor interval time. In some instances, the air may not be monitored (e.g., a dispersion event is initiated at the dispersion interval regardless of air content or quality).

In various embodiments, use of the solution distribution system 10 may result in a pathogen content reduction of 90%. Various different dispersion event periods, dispersion event dosages, and/or dispersion intervals may be used to change the efficiency of the solution distribution system 10 (e.g., the desired efficiency may be different for different application such as residential, commercial, and/or industrial).

The first dispersion event may be caused by the engagement of a control valve (e.g., the dispersion valve assembly 235) to dispense the anti-pathogenic solution via the dispersion nozzle assembly 100 into the air stream for the dispersion event period. For example, the dispersion valve assembly 235 may be moved into delivery mode as discussed above in reference to FIG. 6A. The dispersion valve assembly 235 (e.g., three-way valve) may be engaged between modes by the controller as discussed above.

At the conclusion of the dispersion event period, the control valve may be engaged to block dispensing of the solution via the dispersion nozzle assembly 100. For example, the dispersion valve assembly 235 (e.g., three-way valve) may be moved into the non-operating mode discussed above in reference to FIG. 6A.

As discussed above in reference to FIG. 15, the dispersion event period and/or the dispersion pressure may be selected to deliver the dispersion event dosage of the anti-pathogenic solution between approximately 1.2 fluid ounces to approximately 1.5 fluid ounces. The dispersion event period may be from approximately 6 seconds to approximately 8 seconds.

Referring now to Block 1620 of FIG. 16, the method of dispensing an anti-pathogenic solution into an air stream managed by an air handling system may include causing a second dispersion event via the dispersion nozzle assembly at a dispersion interval. The second dispersion event may provide the dispersion event dosage of anti-pathogenic solution for the dispersion event period.

The second dispersion event may be caused by the engagement of a control valve (e.g., the pick-up valve 130 or the dispersion valve assembly 235 (e.g., three-way valve)) to dispense the anti-pathogenic solution via the dispersion nozzle assembly 100 into the air stream for the dispersion event period. For example, the dispersion valve assembly 235 (e.g., three-way valve) may be moved from the non-operating mode into delivery mode as discussed above in reference to FIG. 6A. At the conclusion of the dispersion event period, the control valve may be engaged to block dispensing of the solution via the dispersion nozzle assembly 100. For example, the dispersion valve assembly 235 (e.g., three-way valve) may be moved into the non-operating mode discussed above in reference to FIG. 6A.

In various embodiments, the dispersion event dosage and/or the dispersion event period of the second dispersion event may be approximately the same as the first dispersion event. In various embodiments, the dispersion event dosage and/or the dispersion event period of the second dispersion event may be different than the first dispersion event (e.g., the first dispersion event dosage may be greater than the second dispersion event dosage).

The dispersion interval may be the amount of time between each dispersion event. For example, the dispersion interval may be approximately 30 minutes to approximately 90 minutes. The dispersion interval may be approximately 60 minutes. In an example embodiment, the solution distribution system 10 may be configured to dispense approximately 1.2 fluid ounces and approximately 1.5 fluid ounces of the solution in 6 seconds, before waiting a dispersion interval of 60 minutes to dispense additional solution.

Referring now to Block 1630 of FIG. 16, the method of dispensing an anti-pathogenic solution into an air stream managed by an air handling system may include causing one or more additional dispersion events at the dispersion interval after the second dispersion event.

In various embodiments, the dispersion interval may be the same between subsequent dispersion events as the dispersion interval between the first dispersion event and the second dispersion event (e.g., a dispersion event may be performed every hour).

In some embodiments, dispersion events may only be carried out only during certain times (e.g., dispersion event timing may be based on the work schedule for an office). For example, dispersion events may only be scheduled for weekdays or more often during weekdays. In various embodiments, the controller 220 discussed herein may be programmable for different environments (e.g., the solution distribution system 10 may have a setting based on days of the week, type of environment, size of the area serviced, and/or the like).

Many modifications and other embodiments of the inventions set forth herein will come to mind to one skilled in the art to which these inventions pertain having the benefit of teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A solution distribution system configured to dispense an anti-pathogenic solution into an air stream managed by an air handling system, the solution distribution system comprising:
a dispersion nozzle assembly structured to dispense the anti-pathogenic solution into the air stream in a dispersion pattern;
a pump module configured to deliver the anti-pathogenic solution to the dispersion nozzle assembly at a dispersion pressure; and
a controller disposed in electrical communication with a control valve and the pump module, wherein the controller is configured to engage the control valve to dispense the anti-pathogenic solution via the dispersion nozzle assembly into the air stream for a dispersion event period and at a dispersion interval.

2. The solution distribution system of claim 1, further comprising a pick-up valve and further defining a solution flow path between the pick-up valve and the dispersion nozzle assembly.

3. The solution distribution system of claim 2, wherein the control valve is a three-way valve connected to a suction hose, a delivery hose, and a bleed hose, and wherein the solution flow path is defined to proceed from the pick-up valve, through the suction hose and delivery hose, and to the dispersion nozzle assembly.

4. (canceled)

5. (canceled)

6. (canceled)

7. The solution distribution system of claim 1, further comprising a fluid pickup assembly configured to draw the anti-pathogenic solution from a solution container and further defining a solution flow path between a pick-up valve and the dispersion nozzle assembly.

8. The solution distribution system of claim 7, wherein the fluid pickup assembly comprises a strainer module and a pick-up valve.

9. The solution distribution system of claim 8, wherein the strainer module of the fluid pickup assembly defines a weighted, cylindrical filter body structured to limit sediment within the solution container from entering the solution flow path.

10. (canceled)

11. (canceled)

12. The solution distribution system of claim 1, wherein the dispersion event period is from approximately 6 seconds to approximately 8 seconds.

13. (canceled)

14. The solution distribution system of claim 1, wherein the dispersion interval is approximately one hour.

15. (canceled)

16. (canceled)

17. The solution distribution system of claim 1, wherein the dispersion nozzle assembly is structured to produce an angular fan dispersion pattern of anti-pathogenic solution.

18. (canceled)

19. The solution distribution system of claim 17, wherein the dispersion nozzle assembly is structured to distribute the anti-pathogenic solution in the angular fan dispersion pattern at a molecule size of between approximately 8 and approximately 15 microns.

20. (canceled)

21. (canceled)

22. (canceled)

23. (canceled)

24. (canceled)

25. A method of assembling a solution distribution system configured to dispense an anti-pathogenic solution into an air stream managed by an air handling system, the method comprising:
providing a dispersion nozzle assembly structured to dispense the anti-pathogenic solution into the air stream in a dispersion pattern;
coupling a pump module to the dispersion nozzle assembly, the pump module being configured to deliver the anti-pathogenic solution to the dispersion nozzle assembly at a dispersion pressure; and
providing a controller disposed in electrical communication with a control valve and the pump module, wherein the controller is configured to engage the control valve to dispense the anti-pathogenic solution via the dispersion nozzle assembly into the air stream for a dispersion event period and at a dispersion interval.

26. The method of claim 25, further comprising providing a pick-up valve coupled to the pump module and further defining a solution flow path between the pick-up valve and the dispersion nozzle assembly.

27. The method of claim 26, wherein the control valve is a three-way valve connected to a suction hose, a delivery hose, and a bleed hose, and wherein the solution flow path is defined to proceed from the pick-up valve, through the suction hose and delivery hose, and to the dispersion nozzle assembly.

28. (canceled)

29. (canceled)

30. (canceled)

31. The method of claim 25 further comprising fluidly coupling a fluid pickup assembly to a solution flow path, wherein the fluid pickup assembly is configured to draw the anti-pathogenic solution from a solution container and the solution flow path is defined between a pick-up valve and the dispersion nozzle assembly.

32. The method of claim 31, wherein the fluid pickup assembly comprises a strainer module and a pick-up valve.

33. The method of claim 32, wherein the strainer module of the fluid pickup assembly defines a weighted, cylindrical filter body structured to limit sediment within the solution container from entering the solution flow path.

34. (canceled)

35. (canceled)

36. The method of claim 25, wherein the dispersion event period is approximately 6 seconds to approximately 8 seconds.

37. (canceled)

38. The method of claim 25, wherein the dispersion interval is approximately one hour.

39. (canceled)

40. (canceled)

41. The method of claim 25, further comprising structuring the dispersion nozzle assembly to produce an angular fan dispersion pattern of anti-pathogenic solution.

42. (canceled)

43. The method of claim 41, wherein the dispersion nozzle assembly is structured to distribute the anti-pathogenic solution in the angular fan dispersion pattern at a molecule size of between approximately 8 and approximately 15 microns.

44-82. (canceled)

83. A dispersion nozzle assembly configured to dispense an anti-pathogenic solution into an air stream managed by an air handling system, the dispersion nozzle assembly comprising:
a dispersion nozzle defining a nozzle outlet structured to dispense the anti-pathogenic solution into the air stream in a dispersion pattern; and
a backflow pressure regulator disposed along a solution flow path to the dispersion nozzle, wherein the backflow pressure regulator is structured to limit air introduction into the solution flow path.

84. The dispersion nozzle assembly of claim 83, wherein the solution flow path is defined between the dispersion nozzle and a pump module, wherein the pump module is configured to provide to the dispersion nozzle at a dispersion pressure.

85. The dispersion nozzle assembly of claim 83, wherein the dispersion nozzle is from approximately 12 inches to approximately 18 inches wide.

86. (canceled)

87. The dispersion nozzle assembly of claim 83, wherein the dispersion pattern is a pulsating spray.

88. (canceled)

89. The dispersion nozzle assembly of claim 84, wherein the dispersion nozzle is fluidly coupled to the pump module via a delivery hose.

90. The dispersion nozzle assembly of claim 83, wherein the dispersion nozzle assembly is in communication with a control valve engaged by a controller configured to engage a control valve to dispense the anti-pathogenic solution via the dispersion nozzle assembly into the air stream for a dispersion event period and at a dispersion interval.

91. The dispersion nozzle assembly of claim 90, wherein the dispersion interval is approximately 60 minutes.

92. (canceled)

93. The dispersion nozzle assembly of claim 90, wherein the dispersion event period is from approximately 6 seconds to approximately 8 seconds.

94. (canceled)

95. (canceled)

96. (canceled)

97. (canceled)

98. The dispersion nozzle assembly of claim 90, wherein the dispersion pattern is an angular fan dispersion pattern of the anti-pathogenic solution.

99. (canceled)

100. The dispersion nozzle assembly of claim 98, wherein the dispersion nozzle assembly is structured to distribute the anti-pathogenic solution in the angular fan dispersion pattern at a molecule size of between approximately 8 and approximately 15 microns.

101. (canceled)

102. (canceled)
